SEQUENCE LISTING (34 sequences)

SEQ ID NO: 1 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 2 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 3 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 4 - protein, 345 aa, Homo sapiens
MYKDCIESTGDYFLLCDAEGPWGIILESLAILGIVVTILLLLAFLFLMRKIQDCSQWNVLPTQLLFLLSVLGLFGLAFAFIIELNQQTAPVRYFLFGVLFALCFSCLLAHASNLVKLVRGCVSFSWTTILCIAIGCSLLQIIIATEYVTLIMTRGMMFVNMTPCQLNVDFVVLLVYVLFLMALTFFVSKATFCGPCENWKQHGRLIFITVLFSIIIWVVWISMLLRGNPQFQRQPQWDDPVVCIALVTNAWVFLLLYIVPELCILYRSCRQECPLQGNACPVTAYQHSFQVENQELSRARDSDGAEEDVALTSYGTPIQPQTVDPTQECFIPQAKLSPQQDAGGV

SEQ ID NO: 5 - protein, 7 aa, Artificial Sequence
  Description of Artificial Sequence Synthetic peptide
SGSYFWG

SEQ ID NO: 6 - protein, 16 aa, Artificial Sequence
  Description of Artificial Sequence Synthetic peptide
SIYYSGITYYNPSLKS

SEQ ID NO: 7 - protein, 11 aa, Artificial Sequence
  Description of Artificial Sequence Synthetic peptide
HDGAVAGLFDY

SEQ ID NO: 8 - protein, 11 aa, Artificial Sequence
  Description of Artificial Sequence Synthetic peptide
GGNNIGSKSVH

SEQ ID NO: 9 - protein, 7 aa, Artificial Sequence
  Description of Artificial Sequence Synthetic peptide
DDSDRPS

SEQ ID NO: 10 - protein, 11 aa, Artificial Sequence
  Description of Artificial Sequence Synthetic peptide
QVWDSSSDHVV

SEQ ID NO: 11 - protein, 121 aa, Artificial Sequence
  Description of Artificial Sequence Synthetic polypeptide
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSS

SEQ ID NO: 12 - protein, 111 aa, Artificial Sequence
  Description of Artificial Sequence Synthetic polypeptide
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQP

SEQ ID NO: 13 - protein, 448 aa, Artificial Sequence
  Description of Artificial Sequence Synthetic polypeptide
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 214 aa, Artificial Sequence
  Description of Artificial Sequence Synthetic polypeptide
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 15 - protein, 5 aa, Artificial Sequence
  Description of Artificial Sequence Synthetic peptide
TYAMN

SEQ ID NO: 16 - protein, 19 aa, Artificial Sequence
  Description of Artificial Sequence Synthetic peptide
RIRSKYNNYATYYAASVKG

SEQ ID NO: 17 - protein, 14 aa, Artificial Sequence
  Description of Artificial Sequence Synthetic peptide
HGNFGNSYVSWFAY

SEQ ID NO: 18 - protein, 14 aa, Artificial Sequence
  Description of Artificial Sequence Synthetic peptide
RSSTGAVTTSNYAN

SEQ ID NO: 19 - protein, 7 aa, Artificial Sequence
  Description of Artificial Sequence Synthetic peptide
GTNKRAP

SEQ ID NO: 20 - protein, 9 aa, Artificial Sequence
  Description of Artificial Sequence Synthetic peptide
ALWYSNLWV

SEQ ID NO: 21 - protein, 125 aa, Artificial Sequence
  Description of Artificial Sequence Synthetic polypeptide
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 22 - protein, 112 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQP

SEQ ID NO: 23 - protein, 452 aa, Artificial Sequence
  Description of Artificial Sequence Synthetic polypeptide
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 24 - protein, 215 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - protein, 5 aa, Artificial Sequence
  Description of Artificial Sequence Synthetic peptide
GYTMN

SEQ ID NO: 26 - protein, 17 aa, Artificial Sequence
  Description of Artificial Sequence Synthetic peptide
LINPYNSDTNYAQKLQG

SEQ ID NO: 27 - protein, 9 aa, Artificial Sequence
  Description of Artificial Sequence Synthetic peptide
VALRVALDY

SEQ ID NO: 28 - protein, 11 aa, Artificial Sequence
  Description of Artificial Sequence Synthetic peptide
KASQNVATHVG

SEQ ID NO: 29 - protein, 7 aa, Artificial Sequence
  Description of Artificial Sequence Synthetic peptide
SASYRYS

SEQ ID NO: 30 - protein, 9 aa, Artificial Sequence
  Description of Artificial Sequence Synthetic peptide
QQYNRYPYT

SEQ ID NO: 31 - protein, 118 aa, Artificial Sequence
  Description of Artificial Sequence Synthetic polypeptide
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSS

SEQ ID NO: 32 - protein, 107 aa, Artificial Sequence
  Description of Artificial Sequence Synthetic polypeptide
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIK

SEQ ID NO: 33 - protein, 445 aa, Artificial Sequence
  Description of Artificial Sequence Synthetic polypeptide
QVQLVQSGAEVKKPGASVKVSCKASGYSFTGYTMNWVRQAPGQGLEWMGLINPYNSDTNYAQKLQGRVTMTTDTSTSTAYMELRSLRSDDTAVYYCARVALRVALDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 34 - protein, 210 aa, Artificial Sequence
  Description of Artificial Sequence Synthetic polypeptide
DIQMTQSPSSLSASVGDRVTITCKASQNVATHVGWYQQKPGKAPKRLIYSASYRYSGVPSRFSGSGSGTEFTLTISNLQPEDFATYYCQQYNRYPYTFGQGTKLEIKKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2022/0373550 A1
(43) Pub. Date: Nov. 24, 2022

(54) METHODS AND COMPOSITIONS FOR MONITORING THE TREATMENT OF RELAPSED AND/OR REFRACTORY MULTIPLE MYELOMA

(71) Applicant: Janssen Biotech, Inc., Horsham, PA (US)

(72) Inventors: Kodandaram Pillarisetti, Spring House, PA (US); Suzette Girgis, Spring House, PA (US); Jenna Goldberg, Raritan, NJ (US); Shun Xin Wang Lin, Spring House, PA (US)

(21) Appl. No.: 17/742,076

(22) Filed: May 11, 2022

Related U.S. Application Data
(60) Provisional application No. 63/187,344, filed on May 11, 2021.

(51) Int. Cl. G01N 33/574

(52) U.S. Cl. CPC G01N 33/57407; G01N 2333/70578

(57) ABSTRACT

Methods of monitoring progression of multiple myeloma or plasmacytoma, particularly relapsed or refractory multiple myeloma, are described. Also described are methods of treating or determining response to a treatment for multiple myeloma or plasmacytoma in a subject.

### CROSS-REFERENCE TO RELATED APPLICATION

This application claims priority to U.S. Provisional Application Ser. No. 63/187,344, filed 11 May 2021, the entire contents of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Apr. 13, 2022, is named PRD4142USNP1_SL.txt and is 36,648 bytes in size.

### FIELD OF THE INVENTION

Methods for monitoring progression or treatment of multiple myeloma, particularly relapsed or refractory multiple myeloma, are disclosed.

### BACKGROUND OF THE INVENTION

Multiple myeloma (MM) is the second most common hematological malignancy and constitutes 2% of all cancer deaths. MM is a heterogeneous disease and caused mostly by chromosome translocations inter alia t(11; 14),t(4; 14),t(8; 14),del(13),del(17) (Drach et al., Blood. 1998; 92(3):802-809, Gertz et al., Blood. 2005; 106(8).2837-2840; Facon et al., Blood. 2001; 97(6): 1566-1571). MM-affected patients can experience a variety of disease-related symptoms due to, bone marrow infiltration, bone destruction, renal failure, immunodeficiency, and the psychosocial burden of a cancer diagnosis. Based on people diagnosed with MM between 2009 and 2015, the 5-year relative survival rate for MM was approximately 51%. This highlights that MM is a difficult-to-treat disease where there are currently insufficient curative options.

Relapsed and refractory MM constitutes a specific unmet medical need. Patients with relapsed and refractory disease are defined as those who achieve minor response or better, then progress while on therapy, or who experience progression within 60 days of their last therapy. Patients who progress after receiving both an immunomodulatory drug and proteasome inhibitor have limited options. Heavily pretreated patients often present with a compromised immune system, which can result in other disease conditions such as opportunistic infections and toxicities (e.g., myelosuppression, peripheral neuropathy, deep vein thrombosis) that persist from prior treatment. Furthermore, patients with advanced MM are often elderly and are susceptible to serious treatment-emergent adverse events (TEAEs) with continued exposure to these therapies. After standard available therapies (such as proteasome inhibitors, immunomodulatory drugs, and monoclonal antibodies) have been exhausted, there is no standard therapy. Selinexor and recently approved BLENREP (belantamab mafodotin-blmf) are licensed in the United States for this highly refractory disease setting. The remaining options for these patients are either entry into a clinical trial, or they can be offered retreatment with a prior treatment regimen (if the toxicity profile for retreatment permits). But often, if no other treatment options remain, they are provided with palliative care to ameliorate disease-related symptoms only. In elderly population, for whom stem cell transplantation is often not a viable option, and in patients with refractory disease who have exhausted all available therapies, the median overall survival is only 8 to 9 months (Kumar et al., Leukemia, 2012, 26:149-157; Usmani et al., Oncolgist, 2016, 21:1355-1361). For patients with disease that is refractory to commonly administered proteasome inhibitors and immunomodulatory drugs, the medium overall survival decreases to only 5 months (Usmani et al., 2016).

Currently available methods for monitoring clinical status and responses to treatment are not optimal for detecting changes rapidly and reliably. For example, monoclonal paraprotein (M-protein) concentration in serum and/or urine is used as an indicator of tumor burden, but the slow rate of change can be problematic when the effects of new therapies for MM need to be assessed quickly (Udd et al., Clin Adv Hematol Oncol. 2017 December; 15(12): 951-961). Serum free light chain (sFLC) is an option with a shorter half-life, but the percentage of patients with MM who have sufficiently elevated levels of sFLCs is low. Measurement of sFLC is also unreliable in patients with renal impairment, a condition which occurs frequently in patients with MM. Bone marrow biopsy is considered the most accurate method of measuring plasma cell infiltration, but is invasive and costly, often underestimates the degree of plasmacytosis, and can result in severe adverse events (Id.).

B-cell maturation antigen (BCMA), also known as CD269 and tumor necrosis factor (TNF) receptor superfamily member 17, is a receptor that plays a critical role in B lymphocytes (B cell) maturation and subsequent differentiation into plasma cells. BCMA binds 2 ligands: A proliferation-inducing ligand (APRIL; CD256) and BAFF (CD257). APRIL and BAFF are type II transmembrane proteins that are readily cleaved by Furin and secreted as soluble trimers by many cells (B cells [autocrine], monocytes, dendritic cells, T cells, osteoclasts, etc.) and can bind to the BCMA receptor. Different from other surface markers, BCMA is exclusively expressed in B-lineage cells and is selectively induced during plasma cell differentiation.

A human BCMA receptor is a 184 amino acid protein that neither has a secretory signal sequence nor any specific protease cleavage site in the N-terminal 54 amino acid extracellular domain. However, the N-terminal fragment is observed as a soluble protein in the serum as a result of gamma secretase activity that cleaves BCMA protein at the transmembrane domain (Laurent et al., Nat Commun. 2015; 6:7333). Inhibition of gamma secretase treatment results in significant increase of BCMA surface protein in human primary B-cells (Laurent et al., 2015, id.). High levels of soluble BCMA (sBCMA) were measured in multiple myeloma patient serum samples (Pillarisetti et al., Blood Adv. 2020 Sep. 22; 4(18): 4538-4549) and correlated with the plasma cell counts (Sanchez et al., Br. J Haematol. 2012; 158(6): 727-738).

BCMA mRNA and protein were universally detected in MM cell lines and in all malignant plasma cells from multiple myeloma patients by Applicants (Pillarisetti et al., Blood Adv. 2020 Sep. 22; 4(18): 4538-4549) and others (Carpenter et al., Clin Cancer Res. 2013; 19(8): 2048-2060; Novak et al., Blood. 2004; 103(2): 689-694). Similarly, in multiple myeloma cell lines and patient samples, BCMA is more stably expressed compared with a key plasma cell marker (CD138) that is also expressed on normal fibroblasts and epithelial cells (Palaiologou et al., Histol Histopathol. 2014; 29(2):177-189). BCMA expression is selective for B cell lineage and was not detected in any major tissues except for infiltrating plasma cells as determined by immunohistochemistry (IHC) methods (Carpenter et al., 2014, id.). Taken together, the selective expression of BCMA on the B cell lineage makes it an appealing target for monitoring disease progression and for T-cell mediated therapy to treat plasma cell disorders like multiple myeloma (Frigyesi et al., Blood. 2014; 123(9): 1336-1340; Tai et al, Immunotherapy. 2015; 7(11): 1187-1199).

There exists a continuing need for improved or alternative methods for monitoring clinical progression and efficacy of therapeutic treatment in MM and plasmacytoma.

### SUMMARY OF THE INVENTION

The application satisfies this need by providing methods of using sBCMA as a surrogate marker of myeloma and plasmacytoma tumor burden, and as a valuable marker for response to a therapy in MM or plasmacytoma patients.

In one aspect, provided herein is a method of monitoring a progression of multiple myeloma in a subject, comprising: (a) measuring a level of sBCMA in a blood sample obtained from the subject; and (b) comparing the level of sBCMA to a reference sBCMA level, wherein the reference sBCMA level is measured from a control blood sample obtained from the subject before the blood sample of (a) is obtained from the subject; wherein an increase in the level of sBCMA compared to the reference sBCMA level indicates one or more of an increased tumor burden or a disease progression, and a decrease in the level of sBCMA compared to the reference sBCMA level indicates one or more of a decreased tumor burden or lack of disease progression.

The disclosure also provides a method of determining a response to a therapy against multiple myeloma in a subject, comprising: (a) treating the subject with the therapy; (b) measuring a level of sBCMA in a blood sample obtained from the subject after the treating of (a); and (c) comparing the level of sBCMA to a reference sBCMA level, wherein the reference sBCMA level is measured from a control blood sample obtained from the subject before the treating of (a); wherein a decrease in the level of sBCMA compared to the reference sBCMA level indicates the subject is responsive to the therapy, and an increase or no change in the level of sBCMA compared to the reference sBCMA level indicates the subject is not responsive to the therapy.

In particular embodiments, the method further comprises treating the subject with a second therapy against multiple myeloma if the level of sBCMA indicates the subject is not responsive to the therapy.

The disclosure also provides a method of treating multiple myeloma or plasmacytoma in a subject in need thereof, comprising: (a) measuring a level of sBCMA in a blood sample obtained from the subject; (b) comparing the level of sBCMA to a reference sBCMA level to measure a tumor burden of the subject; and (c) administering a therapy to the subject based on the tumor burden measured in (b).

In particular embodiments, the method further comprises treating the subject with a therapy against multiple myeloma or plasmacytoma before the blood sample is obtained from the subject, wherein the reference sBCMA level is measured from a control blood sample obtained from the subject before the subject is treated with the therapy, and the treatment comprises: (a) continuing treating the subject with the therapy if the level of sBCMA measured in the blood sample obtained from the subject is lower than the reference sBCMA level, or (b) treating the subject with a second therapy against multiple myeloma or plasmacytoma if the level of sBCMA is the same or higher than the reference sBCMA level.

The disclosure also provides a method of assessing response to teclistamab or talquetamab in a subject with multiple myeloma or plasmacytoma, comprising: (a) treating the subject with teclistamab or talquetamab; (b) measuring a level of sBCMA in a blood sample obtained from the subject after the treating of (a); and (c) comparing the level of sBCMA to a reference sBCMA level, wherein the reference sBCMA level is measured from a control blood sample obtained from the subject before the treating of (a); wherein a decrease in the level of sBCMA compared to the reference sBCMA level indicates the subject is responsive to teclistamab or talquetamab, and an increase or no change in the level of sBCMA compared to the reference sBCMA level indicates the subject is not responsive to teclistamab or talquetamab.

In particular embodiments, the method further comprises treating the subject with a second therapy against multiple myeloma or plasmacytoma if the level of sBCMA indicates the subject is not responsive to teclistamab or talquetamab.

In particular embodiments, the blood sample is obtained from the subject about 4-16 weeks, preferably about 4-12 weeks, such as 4, 5, 6, 7, 8, 9, 10, 11 or 12 weeks, after the subject is treated with the therapy.

In particular embodiments, the therapy comprises a CD3 bispecific antibody. In particular embodiments, the CD3 bispecific antibody is teclistamab or talquetamab. In particular embodiments, the therapy comprises intravenously administering to the subject about 38-720 μg/kg per dose of teclistamab, preferably about 270-720 μg/kg per dose. In other embodiments, the therapy comprises subcutaneously administering to the subject about 80-3000 μg/kg per dose of teclistamab, preferably about 720-3000 μg/kg per dose. In particular embodiments, the therapy comprises intravenously administering to the subject about 0.5-180 μg/kg per dose of talquetamab, preferably about 60-180 μg/kg per dose. In particular embodiments, the therapy comprises subcutaneously administering to the subject about 5-800 μg/kg per dose of talquetamab, preferably about 405-800 μg/kg per dose.

In particular embodiments, the therapy is administered bi-weekly or weekly.

In particular embodiments, the second therapy comprises one or more of autologous stem cell transplants (ASCT), radiation, surgery, chemotherapeutic agents, CAR-T therapies, cellular therapies, immunomodulatory agents, targeted cancer therapies, or combinations thereof.

In particular embodiments, the subject has relapsed and/or refractory multiple myeloma.

In particular embodiments, the blood sample is serum, whole blood, or plasma, preferably serum.

In particular embodiments, the level of sBCMA in the blood sample is measured using an electrochemiluminescence ligand binding assay, an enzyme-linked immunosorbent assay (ELISA), or mass spectrometry.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1A-FIG. 1B show graphs demonstrating the change in sBCMA level from baseline to C3D1 for teclistamab (FIG. 1A) and talquetamab (FIG. 1B) in responders and non-responders. Cycle 3 Day 8 data was used for 3 patients (teclistamab) and 2 patients (talquetamab) who had missing Cycle 3 Day 1 data.

FIG. 2A-FIG. 2B show graphs demonstrating the change in sBCMA level from baseline to C3D1 for teclistamab (FIG. 2A) and talquetamab (FIG. 2B), according to response to treatment. sCR, stringent complete response; CR, complete response; VGPR, very good partial response; PR, partial response; MR, minimal response; SD, stable disease; PD, progressive disease.

FIG. 3A-FIG. 3B show graphs demonstrating the change in sBCMA level over time for teclistamab (FIG. 3A) and talquetamab (FIG. 3B), according to response to treatment.

FIG. 4A-FIG. 4B show graphs demonstrating the change in sBCMA level from baseline to C3D1 for teclistamab (FIG. 4A) and talquetamab (FIG. 4B), according to response to treatment. FIG. 4A includes teclistamab i.v. doses 0.3-720 μg/kg and s.c. doses 80-3000 μg/kg; Cycle 3 Day 8 data used for 3 patients who had missing Cycle 3 Day 1 data; 3 patients with % sBCMA change>500% not shown: 508% (SD), 1201% (SD), 2620% (SD). FIG. 4B includes talquetamab i.v. doses 1-180 μg/kg and s.c. doses 5-800 μg/kg; Cycle 3 Day 8 data used for 2 patients who had missing Cycle 3 Day 1 data. % sBCMA change was calculated as (Cycle 3 Day 1 pre-dose sBCMA Baseline/sBCMA Baseline)×100.

FIG. 5A-FIG. 5D show graphs demonstrating that patients with a high tumor burden responded to teclistamab at a dose of 270-720 μg/kg i.v. or 720-3000 μg/kg s.c. (FIG. 5A-FIG. 5B) and talquetamab at a dose of 60-180 μg/kg i.v. or 405-800 μg/kg s.c. (FIG. 5C-FIG. 5D).

FIG. 6A-FIG. 6B show graphs demonstrating the patient response according to sBCMA level at baseline for teclistamab (FIG. 6A) and talquetamab (FIG. 6B).

FIG. 7A-FIG. 7B show graphs demonstrating the patient response according to tumor burden for teclistamab (FIG. 7A) and talquetamab (FIG. 7B) treatments.

FIG. 8 shows a graph demonstrating the correlation between baseline sBCMA and % bone marrow tumor plasma cells. Data includes patients with both baseline sBCMA and baseline % bone marrow plasma cells; patients with extramedullary plasmacytomas excluded.

FIG. 9A-FIG. 9B show graphs demonstrating that baseline levels of sBCMA were similar in patients with high- and standard-risk cytogenetics, and by Cycle 3 Day 1, teclistamab (FIG. 9A) and talquetamab (FIG. 9B) modulated sBCMA levels in patients with high- and standard-risk cytogenetics. Active doses of teclistamab were 270-720 μg/kg i.v. or 720-3000 μg/kg s.c.; active doses of talquetamab were 60-180 μg/kg i.v. or 405-800 μg/kg s.c.

FIG. 10 shows the percent sBCMA Change from Baseline on Cycle 4 Day 1 by Best Response as Assessed by Independent Review Committee (IRC); Pharmacokinetics Evaluable Analysis Set in the Efficacy Analysis Set (Pivotal RP2D). Key: RP2D=recommended Phase 2 dose; sCR=stringent complete response; CR=complete response; VGPR=very good partial response; PR=partial response; MR=minimal response; SD=stable disease; PD=progressive disease, sBCMA=Soluble B Cell Maturation Antigen.

FIG. 11 shows the percent sBCMA Change from Baseline on Cycle 4 Day 1 by Best Response as Assessed by Investigator; Pharmacokinetics Evaluable Analysis Set in the Efficacy Analysis Set (Phase 1). Key: sCR=stringent complete response; CR=complete response; VGPR=very good partial response; PR=partial response; SD=stable disease; PD=progressive disease; sBCMA=soluble B Cell Maturation Antigen.

### DETAILED DESCRIPTION

The disclosed methods can be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods. All patents, published patent applications and publications cited herein are incorporated by reference as if set fourth fully herein.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “about” when used in reference to numerical ranges, cutoffs, or specific values means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of an assay, result or embodiment, “about” means within one standard deviation per the practice in the art, or a range of up to 10%, whichever is larger.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The term “antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions can be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins can be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species can be assigned to one of two clearly distinct types, namely kappa (κ) and lambda (λ), based on the amino acid sequences of their constant domains.

The terms “antigen binding fragment” or “antigen binding domain” refers to a portion of an immunoglobulin molecule that binds an antigen. Antigen binding fragments can be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains can be linked together via a synthetic linker to form various types of single chain antibody designs where the VH/VL domains can pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

“BCMA” refers to human B-cell maturation antigen, also known as CD269 or TNFRSF17 (UniProt Q02223). The extracellular domain of BCMA encompasses residues 1-54 of Q02223. Human BCMA comprises the amino acid sequence of SEQ ID NO: 1.

SEO ID NO: 1
MLQMAGQCSQNEYFDSLLHACIPCQLRCSSNTPPLTCQRYCNASVTNSVK
GTNAILWTCLGLSLIISLAVFVLMFLLRKINSEPLKDEFKNTGSGLLGMA
NIDLEKSRTGDEIILPRGLEYTVEECTCEDCIKSKPKVDSDHCFPLPAME
EGATILVTTKTNDYCKSLPAALSATEIEKSISAK

“sBCMA”, “soluble BCMA”, and “serum BCMA” refer to the extracellular domain of BCMA (residues 1-57 of SEQ ID NO: 1), which is cleaved from the membrane-bound form on plasma cells by gamma secretase, released into the blood, and solubilized in the serum.

The term “bispecific” refers to an antibody that specifically binds two distinct antigens or two distinct epitopes within the same antigen. The bispecific antibody can have cross-reactivity to other related antigens, for example to the same antigen from other species (homologs), such as human or monkey, for example Macaca cynomolgus (cynomolgus, cyno) or Pan troglodytes, or can bind an epitope that is shared between two or more distinct antigens.

“BCMAxCD3 bispecific antibody” refers to a bispecific antibody that specifically binds BCMA and CD3.

The terms “bind specifically” or “specifically binds” or derivatives thereof when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules. Typically, an antibody binds to a cognate antigen with a Kd of less than about 1×10⁻⁶ M, as measured by a surface plasmon resonance assay or a cell-binding assay. Phrases such as “[antigen]-specific” antibody (e.g., GPRC5D-specific antibody) are meant to convey that the recited antibody specifically binds the recited antigen.

The term “biological marker” or “biomarker” refers to a substance, the change and/or the detection of which indicates a particular biological state. A “biomarker” can indicate a change in the level of polypeptide or protein expression that may correlate with the risk, susceptibility to treatment, or progression of a disease. In some embodiments, the biomarker can be a polypeptide or protein, or a fragment thereof. The relative level of specific proteins can be determined by methods known in the art. For example, antibody-based methods, such as an immunoblot, enzyme-linked immunosorbent assay (ELISA), or other methods can be used. In some embodiments, the indication is the responsiveness of a disease, e.g., a cancer (e.g., MM or plasmacytoma), to a given treatment (e.g., an antibody, such as teclistamab or talquetamab).

The term “cancer” as used herein refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. A “cancer” or “cancer tissue” can include a tumor.

The term “CD3” refers to a human antigen which is expressed on T cells as part of the multimolecular T cell receptor (TCR) complex and which consists of a homodimer or heterodimer formed from the association of two or four receptor chains: CD3 epsilon, CD3 delta, CD3 zeta and CD3 gamma. The term “CD3” includes any CD3 variant, isoform and species homolog which is naturally expressed by cells (including T cells) or can be expressed on cells transfected with genes or cDNA encoding those polypeptides, unless noted. Human CD3 epsilon comprises the amino acid sequence of SEQ ID NO: 2. SEQ ID NO: 3 shows the extracellular domain of human CD3 epsilon.

SEP ID NO: 2
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI
SEQ ID NO: 3
DGNEEMGGITQTPYKVSISGTTVILTCPQYPGSEILWQHNDKNIGGDEDD
KNIGSDEDHLSLKEFSELEQSGYYVCYPRGSKPEDANFYLYLRARVCENC
MEMD

The terms “CH3 region” or “CH3 domain” refer to the CH3 region of an immunoglobulin. The CH3 region of human IgG1 antibody corresponds to amino acid residues 341-446. However, the CH3 region can also be any of the other antibody isotypes as described herein.

The term “combination” as used herein means that two or more therapeutics are administered to a subject together in a mixture, concurrently as single agents or sequentially as single agents in any order.

The term “complementarity determining regions” (CDR) as used herein refers to antibody regions that bind an antigen. CDRs can be defined using various delineations such as Kabat (Wu et al. J Exp Med 132: 211-50, 1970) (Kabat et al., Sequences of Proteins of Immunological Interest, 5th Ed. Public Health Service, National Institutes of Health, Bethesda, Md., 1991), Chothia (Chothia et al. J Mol Biol 196: 901-17, 1987), IMGT (Lefranc et al. Dev Comp Immunol 27: 55-77, 2003) and AbM (Martin and Thornton J Bmol Biol 263: 800-15, 1996). The correspondence between the various delineations and variable region numbering are described (see e.g. Lefranc et al. Dev Comp Immunol 27: 55-77, 2003; Honegger and Pluckthun, J Mol Biol 309:657-70, 2001; International ImMunoGeneTics (IMGT) database; Web resources, http://www_imgt_org). Available programs such as abYsis by UCL Business PLC can be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification

The term “comprising” as used herein is intended to include examples encompassed by the terms “consisting essentially of” and “consisting of”; similarly, the term “consisting essentially of” is intended to include examples encompassed by the term “consisting of.” Unless the context clearly requires otherwise, throughout the description and the claims, the words “comprise”, “comprising”, “having”, and the like are to be construed in an inclusive sense as opposed to an exclusive or exhaustive sense; that is to say, in the sense of “including, but not limited to”.

As used herein, a “control sample” or “control blood sample” refers to a baseline sample or blood sample from a subject who has not been exposed to or treated with a particular therapy, e.g., teclistamab or talquetamab.

The term “enhance” or “enhanced” as used herein refers to an enhancement in a measured level of sBCMA when compared to a control level or reference level. “Enhanced” can be an enhancement of about 10%, 20%, 30%, 40%, 50%, 60%, 70%, 80%, 90%, 100% or more, or an enhancement that is statistically significant.

The term “Fc gamma receptor” (FcγR) as used herein refers to well-known FcγRI, FcγRIIa, FcγRIIb or FcγRIII. Activating FcγR includes FcγRI, FcγRIIa and FcγRIII.

As used herein, the terms “G-protein coupled receptor family C group 5 member D” and “GPRC5D” specifically include the human GPRC5D protein, for example as described in SEQ ID NO: 4 or GenBank Accession No. BC069341, NCBI Reference Sequence: NP_061124.1 and UniProtKB/Swiss-Prot Accession No. Q9NZD1 (see also Brauner-Osborne, H. et al. 2001, Biochim. Biophys. Acta 1518, 237-248).

SEQ ID NO: 4
MYKDCIESTGDYFLLCDAEGPWGIILESLAILGIVVTILLLLAFLFLMRK
IQDCSQWNVLPTQLLFLLSVLGLFGLAFAFIIELNQQTAPVRYFLFGVLF
ALCFSCLLAHASNLVKLVRGCVSFSWTTILCIAIGCSLLQIIIATEYVTL
IMTRGMMFVNMTPCQLNVDFVVLLVYVLFLMALTFFVSKATFCGPCENWK
QHGRLIFITVLFSIIIWVVWISMLLRGNPQFQRQPQWDDPVVCIALVTNA
WVFLLLYIVPELCILYRSCRQECPLQGNACPVTAYQHSFQVENQELSRAR
DSDGAEEDVALTSYGTPIQPQTVDPTQECFIPQAKLSPQQDAGGV

As used herein, a “GPRC5D×CD3 antibody” is a multispecific antibody, optionally a bispecific antibody, which comprises two different antigen-binding regions, one of which binds specifically to the antigen GPRC5D and one of which binds specifically to CD3.

The term “human antibody” as used herein refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” can contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

The term “humanized antibody” as used herein refers to an antibody in which at least one CDR is derived from non-human species and at least one framework is derived from human immunoglobulin sequences. Humanized antibody can include substitutions in the frameworks so that the frameworks cannot be exact copies of expressed human immunoglobulin or human immunoglobulin germline gene sequences.

The term “isolated” as used herein refers to a homogenous population of molecules (such as synthetic polynucleotides or a protein such as an antibody) which have been substantially separated and/or purified away from other components of the system the molecules are produced in, such as a recombinant cell, as well as a protein that has been subjected to at least one purification or isolation step. “Isolated antibody” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

The term “monoclonal antibody” as used herein refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies can have heterogeneous glycosylation within the antibody population. Monoclonal antibodies can be monospecific or multispecific such as bispecific, monovalent, bivalent or multivalent.

The term “mutation” as used herein refers to an engineered or naturally occurring alteration in a polypeptide or polynucleotide sequence when compared to a reference sequence. The alteration can be a substitution, insertion or deletion of one or more amino acids or polynucleotides.

The term “multispecific” as used herein refers to an antibody that specifically binds at least two distinct antigens or at least two distinct epitopes within the same antigen. Multispecific antibody can bind for example two, three, four or five distinct antigens or distinct epitopes within the same antigen.

Current IMWG (International Myeloma Working Group) guidelines define “negative minimal residual disease status” or “negative MRD status” or “MRD negative” as fewer than one tumor cell in 100000 bone marrow cells (10⁻⁵) in a patient who fulfills the criteria for complete response (CR). Negative minimal residual disease status can be determined using next generation sequencing (NGS).

The term “pharmaceutical composition” as used herein refers to composition that comprises an active ingredient and a pharmaceutically acceptable carrier.

The term “pharmaceutically acceptable carrier” or “excipient” as used herein refers to an ingredient in a pharmaceutical composition, other than the active ingredient, which is nontoxic to a subject.

The term “recombinant” as used herein refers to nucleic acids, antibodies and other proteins or peptides that are prepared, expressed, created or isolated by recombinant methods. For example, segments from different sources can be joined to produce recombinant DNA, RNA, antibodies or proteins.

The term “reduce” or “reduced” as used herein refers to a reduction in a measured level of sBCMA when compared to a control level or reference level. “Reduced” can be a reduction of about 10%, 20%, 30%, 40%, 50%, 60%, 70%, 80%, 90%, 100% or more, or a reduction that is statistically significant.

The term “reference level” as used herein refers to a level of sBCMA that is an absolute level; a relative level; a level that has an upper and/or lower limit; a range of levels; an average level; a median level, a mean level, or a level as compared to a particular control, baseline, or test level. A reference level of sBCMA can be based on an individual sample level, such as for example, a level obtained from a sample from a subject with MM or plasmacytoma, but at an earlier point in time, or a level obtained from a sample from an MM subject or subject with plasmacytoma other than the individual being tested, or a “normal” subject, that is an individual not diagnosed with MM or plasmacytoma. The reference level can be based on a large number of samples, such as from MM or plasmacytoma patients or normal individuals or based on a pool of samples including or excluding the sample to be tested.

The term “refractory” as used herein refers to a cancer that is not amendable to surgical intervention and is initially unresponsive to therapy.

The term “relapsed” as used herein refers to a cancer that responded to treatment but then returns.

The term “response”, “responsiveness” or “responsive” when used in reference to a treatment or therapy refers to the degree of effectiveness of the treatment or therapy in lessening or decreasing the symptoms of a disease being treated. The disease can be, e.g., MM or plasmacytoma. For example, the term “increased responsiveness” when used in reference to a treatment of a cell or a subject refers to an increase in the effectiveness in lessening or decreasing the symptoms of the disease when measured using any methods known in the art. In certain embodiments, the increase in the effectiveness is at least about 5%, at least about 10%, at least about 20%, at least about 30%, at least about 40%, or at least about 50%.

As used herein, a “sample” is intended to include any sampling of cells, tissues, or bodily fluids in which expression of a gene, protein, or biomarker can be detected. Examples of such samples include, but are not limited to, biopsies, smears, blood, lymph, urine, saliva, or any other bodily secretion or derivative thereof. Blood can, for example, include whole blood, plasma, serum, or any derivative of blood. Samples can be treated, for example with an anti-coagulant, or untreated. Samples can be obtained from a subject by a variety of techniques, which are known to those skilled in the art.

The term “subject” as used herein includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

The term “T cell redirecting therapeutic” as used herein refers to a molecule containing two or more binding regions, wherein one of the binding regions specifically binds a cell surface antigen on a target cell or tissue and wherein a second binding region of the molecule specifically binds a T cell antigen. Examples of cell surface antigen include a tumor associated antigen, such as BCMA or GPRC5D. Examples of T cell antigen include, e.g., CD3. This dual/multi-target binding ability recruits T cells to the target cell or tissue leading to the eradication of the target cell or tissue.

The term “therapeutically effective amount” as used herein refers to an amount effective, at doses and for periods of time necessary, to achieve a desired therapeutic result. A therapeutically effective amount can vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

The term “treat” or “treatment” as used herein refers to both therapeutic treatment and prophylactic or preventative measures, wherein the object is to prevent or slow down (lessen) an undesired physiological change or disorder. Beneficial or desired clinical results include alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if a subject was not receiving treatment. Those in need of treatment include those already with the condition or disorder as well as those prone to have the condition or disorder or those in which the condition or disorder is to be prevented.

The term “tumor burden” or “tumor load” as used herein refers to the number of tumor cells, the size of a tumor, the total mass of tumor tissue, or the amount of cancer in the body of a subject.

The term “tumor cell” or “cancer cell” as used herein refers to a cancerous, pre-cancerous or transformed cell, either in vivo, ex vivo, or in tissue culture, that has spontaneous or induced phenotypic changes. These changes do not necessarily involve the uptake of new genetic material. Although transformation can arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples. The application contemplates use of any of the applicable components and/or steps in any combination that can be used the application, whether or not a particular combination is expressly described.

sBCMA and Methods of Use

The methods provided herein are based, in part, on the finding that a detectable decrease or increase in serum BCMA (sBCMA) level is observed in subjects with multiple myeloma or plasmacytoma who are responsive and non-responsive, respectively, to a given treatment (e.g., an antibody, such as teclistamab or talquetamab), the level of sBCMA can be used as a biomarker for predicting or monitoring the responsiveness of the subjects to the treatment, and/or the progression of the cancer in the subjects.

Accordingly, in one general aspect, the disclosure relates to a method of monitoring a progression of a cancer in a subject, comprising: (a) measuring a level of sBCMA in a blood sample obtained from the subject; and (b) comparing the level of sBCMA to a reference sBCMA level, wherein the reference sBCMA level is measured from a control blood sample obtained from the subject before the blood sample of (a) is obtained from the subject; wherein an increase in the level of sBCMA compared to the reference sBCMA level indicates one or more of an increased tumor burden or a disease progression, and a decrease in the level of sBCMA compared to the reference sBCMA level indicates one or more of a decreased tumor burden or lack of disease progression. In addition, sBCMA may account for plasmacytoma, for example, patients with plasmacytoma may have low tumor burden measured by % bone marrow plasma cell (BMPC) but high sBCMA level. Preferably, the cancer is multiple myeloma (MM) or plasmacytoma, more preferably the cancer is relapsed and/or refractory multiple myeloma.

In some embodiments, the level of sBCMA can be measured after a period of time from the measurement of the reference sBCMA level in the control blood sample, such as, for example, about 4-16 weeks after, about 2-6 months after, about 4-12 months after, or longer, after the measurement of the reference sBCMA level. In some embodiments, the level of sBCMA is measured more than once after the measurement of the reference sBCMA level in the control blood sample, to determine the progression of the cancer in the subject. In some embodiments, the level of sBCMA can be measured at multiple timepoints to determine the progression of the cancer in the subject over time. For example, the level of sBCMA can be measured once per day, once per week, once per month, once every six months, once per year, or any length of time in between, in order to determine the progression of the cancer in the subject.

In another general aspect, the disclosure relates to a method of determining a response to a therapy against multiple myeloma in a subject, comprising: (a) treating the subject with the therapy; (b) measuring a level of sBCMA in a blood sample obtained from the subject after the treating of (a); and (c) comparing the level of sBCMA to a reference sBCMA level, wherein the reference sBCMA level is measured from a control blood sample obtained from the subject before the treating of (a); wherein a decrease in the level of sBCMA compared to the reference sBCMA level indicates the subject is responsive to the therapy, and an increase or no change in the level of sBCMA compared to the reference sBCMA level indicates the subject is not responsive to the therapy.

In some embodiments, the multiple myeloma is relapsed and/or refractory multiple myeloma.

In some embodiments, the blood sample is obtained from the subject 4-16 weeks, preferably 4-12 weeks, such as 4, 5, 6, 7, 8, 9, 10, 11 or 12 weeks, after the subject is treated with the therapy. In some embodiments, the blood sample is obtained from the subject about 2-6 months after, about 4-12 months after, or longer, after the subject is treated with the therapy. In some embodiments, the level of sBCMA is measured more than once after the measurement of the reference sBCMA level in the control blood sample. In some embodiments, the level of sBCMA can be measured at multiple timepoints to determine the response to the therapy over time. For example, the level of sBCMA can be measured once per day, once per week, once per month, once every six months, once per year, or any length of time in between, in order to determine the response to the therapy over time.

In some embodiments, the blood sample is whole blood, serum, or plasma, preferably serum. The blood sample can be treated with, e.g., an anti-coagulant, or untreated.

In some embodiments, the therapy is a CD3 bispecific antibody. In some embodiments, the therapy is teclistamab or talquetamab. In some embodiments, the therapy is a CAR-T therapy. In some embodiments, the method comprises treating the subject with a second therapy against multiple myeloma if the level of sBCMA is increased or not changed compared to the reference sBCMA level. In some embodiments, the second therapy is a CD3 bispecific antibody. In some embodiments, the second therapy is teclistamab or talquetamab. In some embodiments, the second therapy is one or more of autologous stem cell transplants (ASCT), radiation, surgery, chemotherapeutic agents, CAR-T therapies, cellular therapies, immunomodulatory agents, targeted cancer therapies, or combinations thereof.

In another general aspect, the disclosure relates to a method of treating multiple myeloma or plasmacytoma in a subject in need thereof, comprising: (a) measuring a level of sBCMA in a blood sample obtained from the subject; (b) comparing the level of sBCMA to a reference sBCMA level to measure a tumor burden of the subject; and (c) administering a therapy to the subject based on the tumor burden measured in (b). In some embodiments, the method further comprises treating the subject with a therapy against multiple myeloma or plasmacytoma before the blood sample is obtained from the subject, wherein the reference sBCMA level is measured from a control blood sample obtained from the subject before the subject is treated with the therapy, and the treatment comprises: (a) continuing treating the subject with the therapy if the level of sBCMA measured in the blood sample obtained from the subject is lower than the reference sBCMA level, or (b) treating the subject with a second therapy against multiple myeloma or plasmacytoma if the level of sBCMA is the same or higher than the reference sBCMA level.

In some embodiments, the multiple myeloma or plasmacytoma is relapsed and/or refractory.

In some embodiments, the blood sample is obtained from the subject 4-16 weeks, preferably 4-12 weeks, such as 4, 5, 6, 7, 8, 9, 10, 11 or 12 weeks, after the subject is treated with the therapy. In some embodiments, the blood sample is whole blood, serum, or plasma, preferably serum. The blood sample can be treated with, e.g., an anti-coagulant, or untreated.

In some embodiments, the therapy is a CD3 bispecific antibody. In some embodiments, the therapy is teclistamab or talquetamab. In some embodiments, the therapy is a CAR-T therapy. In some embodiments, the second therapy is a CD3 bispecific antibody. In some embodiments, the second therapy is teclistamab or talquetamab. In some embodiments, the second therapy is one or more of autologous stem cell transplants (ASCT), radiation, surgery, chemotherapeutic agents, CAR-T therapies, cellular therapies, immunomodulatory agents, targeted cancer therapies, or combinations thereof.

In some embodiments, the reference sBCMA level is a pre-determined level of sBCMA, and the treatment comprises treating the subject with a therapy against multiple myeloma or plasmacytoma if the level of sBCMA is lower than the pre-determined level. The pre-determined level of sBCMA can vary, depending on the therapy used. A pre-determined level for a therapy can be determined based on the responsiveness of an individual to the therapy and saved as part of the medical record of the individual. A pre-determined level for a therapy can be determined based on the average responsiveness of multiple individuals to the therapy. In some embodiments, the pre-determined level of sBCMA, preferably for a CD3 bispecific antibody, is about 400-1000 ng/mL, such as about 400 ng/mL, about 500 ng/mL, about 600 ng/mL, about 700 ng/mL, about 800 ng/mL, about 900 ng/mL, or about 1000 ng/mL. Preferably the pre-determined level of sBCMA for teclistamab or talquetamab is about 400-800 ng/mL, more preferably about 400-600 ng/mL, such as about 400, about 450, about 500, about 550 or about 600 ng/ml.

In another general aspect, the disclosure relates to a method of assessing response to teclistamab or talquetamab in a subject with multiple myeloma or plasmacytoma, comprising: (a) treating the subject with teclistamab or talquetamab; (b) measuring a level of sBCMA in a blood sample obtained from the subject after the treating of (a); and (c) comparing the level of sBCMA to a reference sBCMA level, wherein the reference sBCMA level is measured from a control blood sample obtained from the subject before the treating of (a); wherein a decrease in the level of sBCMA compared to the reference sBCMA level indicates the subject is responsive to teclistamab or talquetamab, and an increase or no change in the level of sBCMA compared to the reference sBCMA level indicates the subject is not responsive to teclistamab or talquetamab. In some embodiments, the method further comprises treating the subject with a second therapy against multiple myeloma or plasmacytoma if the level of sBCMA indicates the subject is not responsive to teclistamab or talquetamab.

In some embodiments, the multiple myeloma or plasmacytoma is relapsed and/or refractory.

In some embodiments, the blood sample is obtained from the subject 4-16 weeks, preferably 4-12 weeks, such as 4, 5, 6, 7, 8, 9, 10, 11 or 12 weeks, after the subject is treated with the therapy. In some embodiments, the blood sample is whole blood, serum, or plasma, preferably serum. The blood sample can be treated with, e.g., an anti-coagulant, or untreated.

A method of the application can be used to assess the response to any cancer therapy in view of the present disclosure. In some embodiments, the therapy is a CD3 bispecific antibody. In some embodiments, the therapy is teclistamab or talquetamab. In some embodiments, the therapy is a CAR-T therapy. In some embodiments, the second therapy is a CD3 bispecific antibody. In some embodiments, the second therapy is teclistamab or talquetamab. In some embodiments, the therapy or second therapy is one or more of autologous stem cell transplants (ASCT), radiation, surgery, chemotherapeutic agents, CAR-T therapies, cellular therapies, immunomodulatory agents, targeted cancer therapies, or combinations thereof, provided that the second therapy is different from the therapy.

Any suitable method can be used to measure the level of sBCMA in view of the present disclosure. In some embodiments of the various methods provided herein, the level (e.g., expression) of sBCMA is determined by measuring the protein level in a sample.

In some embodiments, the sample is obtained from a biopsy, smear, blood, lymph, urine, saliva, or any other bodily secretion or derivative thereof from a subject. In preferred embodiments, the sample is a blood sample. A blood sample can, for example, include whole blood, plasma, serum, or any derivative of blood. Preferably, the blood sample is serum. Samples can be untreated, or can be treated or processed according to methods known in the art, for example, with an anti-coagulant. Preferably the sample is untreated.

In certain embodiments, the level (e.g., expression) of the biomarker is measured by electrochemiluminescence ligand binding assay or other similar methods known in the art. In certain embodiments, the level (e.g., expression) of the biomarker is measured by enzyme-linked immunosorbent assay-based methodologies (ELISA) or other similar methods known in the art. An ELISA can use one or several different anti-BCMA antibodies. Non-limiting examples of commercially available antibodies that can be used in ELISA are MAB193 (R&D Systems), Vicky-1 (Novus Biologicals; Cat. No. NBP1-97637), LS-B2728 (LifeSpan Biosciences), or BCMA/2366 (NSJ Bioreagents; Cat. No. V3814). In certain embodiments, the level (e.g., expression) of the biomarker is measured by exposing the sample to a mass analysis technique (e.g., mass spectrometry) or other similar methods known in the art.

In certain embodiments, reagents are provided for the detection and/or quantification of biomarker proteins. The reagents can include, but are not limited to, primary antibodies that bind the protein biomarkers, secondary antibodies that bind the primary antibodies, affibodies that bind the protein biomarkers, aptamers (e.g., a SOMAmer) that bind the protein or nucleic acid biomarkers (e.g., RNA or DNA), and/or nucleic acids that bind the nucleic acid biomarkers (e.g., RNA or DNA). The detection reagents can be labeled (e.g., fluorescently) or unlabeled. Additionally, the detection reagents can be free in solution or immobilized.

In certain embodiments, the level of one or more additional biomarkers are monitored simultaneously or sequentially. Multiple biomarkers can be monitored simultaneously or sequentially.

In certain embodiments, when quantifying the level of a biomarker(s) present in a sample, the level can be determined on an absolute basis or a relative basis. When determined on a relative basis, comparisons can be made to controls, which can include, but are not limited to, historical samples from the same patient (e.g., a series of samples over a certain time period), level(s) found in a subject or population of subjects without the disease or disorder (e.g., MM), a threshold value, and an acceptable range.

Another aspect of the application relates to kits or combinations of reagents useful for methods of the invention, comprising one or more agents for measuring the level of sBCMA in a blood sample. The reagents can include, but are not limited to, primary antibodies that bind the protein biomarkers, secondary antibodies that bind the primary antibodies, affibodies that bind the protein biomarkers, aptamers (e.g., a SOMAmer) that bind the protein or nucleic acid biomarkers (e.g., RNA or DNA), and/or nucleic acids that bind the nucleic acid biomarkers (e.g., RNA or DNA). The detection reagents can be labeled (e.g., fluorescently) or unlabeled. Additionally, the detection reagents can be free in solution or immobilized.

Kits can include all components necessary or sufficient for assays, which can include, but is not limited to, detection reagents (e.g., probes), buffers, control reagents (e.g., positive and negative controls), amplification reagents, solid supports, labels, instruction manuals, etc. In certain embodiments, the kit comprises a set of probes for detecting sBCMA, optionally in combination with one or more additional biomarkers, and a solid support to immobilize the set of probes. In certain embodiments, the kit comprises a set of probes for sBCMA, optionally in combination with probes for one or more additional biomarkers, a solid support, and reagents for processing the sample to be tested (e.g., reagents to isolate the protein or nucleic acids from the sample).

### Cancers

Methods of the application can be used to treat or monitor a cancer, preferably a hematological malignancy or plasma cell proliferative disorder, more preferably a relapsed or refractory hematological malignancy or plasma cell proliferative disorder.

In some embodiments, the hematological malignancy is a multiple myeloma, a smoldering multiple myeloma, a monoclonal gammopathy of undetermined significance (MGUS), an acute lymphoblastic leukemia (ALL), a diffuse large B-cell lymphoma (DLBCL), a Burkitt's lymphoma (BL), a follicular lymphoma (FL), a mantle-cell lymphoma (MCL), Waldenstrom's macroglobulinema, a plasma cell leukemia, a light chain amyloidosis (AL), a precursor B-cell lymphoblastic leukemia, a precursor B-cell lymphoblastic leukemia, an acute myeloid leukemia (AML), a myelodysplastic syndrome (MDS), a chronic lymphocytic leukemia (CLL), a B cell malignancy, a chronic myeloid leukemia (CML), a hairy cell leukemia (HCL), a blastic plasmacytoid dendritic cell neoplasm, Hodgkin's lymphoma, non-Hodgkin's lymphoma, a marginal zone B-cell lymphoma (MZL), a mucosa-associated lymphatic tissue lymphoma (MALT), plasma cell leukemia, anaplastic large-cell lymphoma (ALCL), leukemia or lymphoma.

In some embodiments, the plasma cell proliferative disorder is asymptomatic myeloma (smoldering multiple myeloma or indolent myeloma), plasmacytomas (e.g., plasma cell dyscrasia, solitary myeloma, solitary plasmacytoma, extramedullary plasmacytoma, and multiple plasmacytoma), monoclonal gammapathy of undetermined significance (MGUS), Waldenstrom's macroglobulinemia, systemic amyloid light chain amyloidosis, and POEMS syndrome (also known as Crow-Fukase syndrome, Takatsuki disease, and PEP syndrome).

In preferred embodiments, the hematological malignancy or plasma cell proliferative disorder is multiple myeloma or plasmacytoma. In some embodiments, the subject has a newly diagnosed multiple myeloma or plasmacytoma. In some embodiments, the subject is relapsed or refractory to treatment with a prior anti-cancer therapeutic, such as a therapeutic used to treat multiple myeloma or other hematological malignancies or plasmacytoma.

In some embodiments, the subject is refractory or relapsed to one or more prior anti-cancer treatments or therapies. Exemplary prior anti-cancer treatments or therapies, include, without limitation, THALOMID® (thalidomide), REVLIMID® (lenalidomide), POMALYST® (pomalidomide), VELCADE® (bortezomib), NINLARO (ixazomib), KYPROLIS® (carfilzomib), FARADYK® (panobinostat), AREDIA® (pamidronate), ZOMETA® (zoledronic acid), DARZALEX® (daratumumab), EMPLICITI® (elotuzumab), melphalan, Xpovio® (Selinexor), BLENREP (belantamab mafodotin-blmf), Venclexta® (Venetoclax), CAR-T therapies, other BCMA-directed therapies, other CD38-directed therapies, or any combinations thereof.

Various qualitative and/or quantitative methods can be used to determine relapse or refractory nature of the disease. According to NCCN Guidelines, “clinical relapse” are defined as having one of more of the following occurred: there are direct signs of cancer growth, signs of organ damage, an increase in the number of size (at least 50% larger) of plasmacytomas or bone lesions, increased calcium levels, an increase in creatinine levels in blood, or a decrease in the number of red blood cells, and “relapse from complete response” is defined as having one or more of the following occurred in a patient who had a complete response: a return of M-proteins in blood or urine, or other signs of myeloma but not meeting the criteria for a clinical relapse progressive disease. (“Progressive disease” is defined as having one or more of the following occurred: at least 25% increase in the amount of M-proteins in the blood or urine, a 25% increase in the number of plasma cells in the bone marrow, an increase in the size or number of bone lesions, or an increase in calcium levels not explained by other conditions).

In some embodiments, the multiple myeloma or plasmacytoma is relapsed or refractory to treatment with an anti-CD38 antibody, selinexor, venetoclax, lenalinomide, bortezomib, pomalidomide, carfilzomib, elotozumab, ixazomib, melphalan or thalidomide, or any combination thereof.

In some embodiments, the multiple myeloma is a high-risk multiple myeloma. Subjects with high-risk multiple myeloma are known to relapse early and have poor prognosis and outcome. Subjects can be classified as having high-risk multiple myeloma is they have one or more of the following cytogenetic abnormalities: t(4; 14)(p16;q32), t(14; 16)(q32;q23), del17p, 1qAmp, t(4; 14)(p16;q32) and t(14; 16)(q32;q23), t(4; 14)(p16;q32) and del17p, t(14; 16)(q32;q23) and del17p, ort(4; 14)(p16;q32), t(14; 16)(q32;q23) and del17p. In some embodiments, the subject having the high-risk multiple myeloma has one or more chromosomal abnormalities comprising: t(4; 14)(p16;q32), t(14; 16)(q32;q23), del17p, 1qAmp, t(4; 14)(p16;q32) and t(14; 16)(q32;q23), t(4; 14)(p16;q32) and del17p, t(14; 16)(q32;q23) and del17p; or t(4:14)(p16;q32), t(14; 16)(q32;q23) and del17p, or any combination thereof.

The cytogenetic abnormalities can be detected for example by fluorescent in situ hybridization (FISH). In chromosomal translocations, an oncogene is translocated to the IgH region on chromosome 14q32, resulting in dysregulation of these genes. t(4; 14)(p16;q32) involves translocation of fibroblast growth factor receptor 3 (FGFR3) and multiple myeloma SET domain containing protein (MMSET) (also called WHSC1/NSD2), and t(14; 16)(q32;q23) involves translocation of the MAF transcription factor C-MAF. Deletion of 17p (del17p) involves loss of the p53 gene locus.

Chromosomal rearrangements can be identified using well known methods, for example fluorescent in situ hybridization, karyotyping, pulsed field gel electrophoresis, or sequencing.

### Treatments

The use of anti-BCMA antibodies for the treatment of lymphomas and multiple myeloma is mentioned in WO2002066516 and WO2010104949. Antibodies against BCMA are described, e.g. in Gras M-P. et al. Int Immunol. 1997; 7:1093-1106, WO200124811, and WO200124812. Bispecific antibodies against BCMA and CD3 are described e.g. in WO2017/031104. Teclistamab and talquetamab are CD3 bispecific antibodies that have been developed to recruit CD3⁺ T-cells to BCMA⁺ or GPRC5D multiple myeloma (MM) cells, respectively.

Anti-BCMA/anti-CD3 antibody teclistamab (also called JNJ-64007957, JNJ-957 or JNJ-7957) (described in WO2017031104A 1, the content of which is incorporated herein by reference in its entirety) was made by Janssen Pharmaceuticals. Teclistamab comprises a BCMA binding arm BCMB69 and a CD3 binding arm CD3B219, the amino acid sequences of which are shown in Table 1 and Table 2, respectively.

Overexpression of GPRC5D in the bone marrow is associated with poor prognosis in patients with multiple myeloma (see e.g., Atamaniuk et al., Eur. J. Clin. Invest. 42:953-960(2012)). This exclusive expression of GPRC5D on the plasma-cell lineage designates it as an ideal target for antimyeloma antibodies. Anti-GPRC5D antibodies and bispecific antibodies against GPRC5D and CD3 are described, e.g., in U.S. Pat. No. 10,562,968, the content of which is incorporated herein by reference in its entirety.

A fully humanized IgG4 anti-GPRC5D/anti-CD3 bispecific antibody, talquetamab (described in U.S. Pat. No. 10,562,968, the content of which is incorporated herein by reference in its entirety), was made by Janssen Pharmaceuticals. It was produced by cultivation of recombinant Chinese Hamster Ovary cells followed by isolation, chromatographic purification, and formulation. Talquetamab comprises a GPRC5D binding arm GC5B596 and a CD3 binding arm CD3B219, the amino acid sequences of which are shown in Table 3 and Table 2, respectively.

TABLE 1
Sequences of BCMA binding arm of Teclistamab

| | | | SEQ |
|---|---|---|---|
| | | | ID |
| | Region | Sequence | NO: |
| BCMB69 | HCDR1 | SGSYFWG | 5 |
| | HCDR2 | SIYYSGITYYNPSLKS | 6 |
| | HCDR3 | HDGAVAGLFDY | 7 |
| | LCDR1 | GGNNIGSKSVH | 8 |
| | LCDR2 | DDSDRPS | 9 |
| | LCDR3 | QVWDSSSDHVV | 10 |
| | VH | QLQLQESGPGLVKPSETLSLTCTVSGGSISS | 11 |
| | | GSYFWGWIRQPPGKGLEWIGSIYYSGITYYN | |
| | | PSLKSRVTISVDTSKNQFSLKLSSVTAADTA | |
| | | VYYCARHDGAVAGLFDYWGQGTLVTVSS | |
| | VL | SYVLTQPPSVSVAPGQTARITCGGNNIGSKS | 12 |
| | | VHWYQQPPGQAPVVVVYDDSDRPSGIPERFS | |
| | | GSNSGNTATLTISRVEAGDEAVYYCQVWDSS | |
| | | SDHVVFGGGTKLTVLGQP | |
| | HC | QLQLQESGPGLVKPSETLSLTCTVSGGSISS | 13 |
| | | GSYFWGWIRQPPGKGLEWIGSIYYSGITYYN | |
| | | PSLKSRVTISVDTSKNQFSLKLSSVTAADTA | |
| | | VYYCARHDGAVAGLFDYWGQGTLVTVSSAST | |
| | | KGPSVFPLAPCSRSTSESTAALGCLVKDYFP | |
| | | EPVTVSWNSGALTSGVHTFPAVLQSSGLYSL | |
| | | SSVVTVPSSSLGTKTYTCNVDHKPSNTKVDK | |
| | | RVESKYGPPCPPCPAPEAAGGPSVFLFPPKP | |
| | | KDTLMISRTPEVTCVVVDVSQEDPEVQFNWY | |
| | | VDGVEVHNAKTKPREEQFNSTYRVVSVLTVL | |
| | | HQDWLNGKEYKCKVSNKGLPSSIEKTISKAK | |
| | | GQPREPQVYTLPPSQEEMTKNQVSLTCLVKG | |
| | | FYPSDIAVEWESNGQPENNYKTTPPVLDSDG | |
| | | SFFLYSRLTVDKSRWQEGNVFSCSVMHEALH | |
| | | NHYTQKSLSLSLGK | |
| | LC | SYVLTQPPSVSVAPGQTARITCGGNNIGSKS | 14 |
| | | VHWYQQPPGQAPVVVVYDDSDRPSGIPERFS | |
| | | GSNSGNTATLTISRVEAGDEAVYYCQVWDSS | |
| | | SDHVVFGGGTKLTVLGQPKAAPSVTLFPPSS | |
| | | EELQANKATLVCLISDFYPGAVIVAWKGDSS | |
| | | PVKAGVETTTPSKQSNNKYAASSYLSLTPEQ | |
| | | WKSHRSYSCQVTHEGSTVEKTVAPTECS | |

TABLE 2
Sequences of CD3 binding arm of Teclistamab and
Talquetamab

| | | | SEQ |
|---|---|---|---|
| | | | ID |
| | Region | Sequence | NO: |
| CD3B219 | HCDR1 | TYAMN | 15 |
| | HCDR2 | RIRSKYNNYATYYAASVKG | 16 |
| | HCDR3 | HGNFGNSYVSWFAY | 17 |
| | LCDR1 | RSSTGAVTTSNYAN | 18 |
| | LCDR2 | GTNKRAP | 19 |
| | LCDR3 | ALWYSNLWV | 20 |
| | VH | EVQLVESGGGLVQPGGSLRLSCAASGFTFNT | 21 |
| | | YAMNWVRQAPGKGLEWVARIRSKYNNYAT | |
| | | YYAASVKGRFTISRDDSKNSLYLQMNSLKTE | |
| | | DTAVYYCARHGNFGNSYVSWFAYWGQGTL | |
| | | VTVSS | |
| | VL | QTVVTQEPSLTVSPGGTVTLTCRSSTGAVTT | 22 |
| | | SNYANWVQQKPGQAPRGLIGGTNKRAPGTP | |
| | | ARFSGSLLGGKAALTLSGVQPEDEAEYYCAL | |
| | | WYSNLWVFGGGTKLTVLGQP | |
| | HC | EVQLVESGGGLVQPGGSLRLSCAASGFTFNT | 23 |
| | | YAMNWVRQAPGKGLEWVARIRSKYNNYAT | |
| | | YYAASVKGRFTISRDDSKNSLYLQMNSLKTE | |
| | | DTAVYYCARHGNFGNSYVSWFAYWGQGTL | |
| | | VTVSSASTKGPSVFPLAPCSRSTSESTAALG | |
| | | CLVKDYFPEPVTVSWNSGALTSGVHTFPAVL | |
| | | QSSGLYSLSSVVTVPSSSLGTKTYTCNVDHK | |
| | | PSNTKVDKRVESKYGPPCPPCPAPEAAGGPS | |
| | | VFLFPPKPKDTLMISRTPEVTCVVVDVSQED | |
| | | PEVQFNWYVDGVEVHNAKTKPREEQFNSTY | |
| | | RVVSVLTVLHQDWLNGKEYKCKVSNKGLPS | |
| | | SIEKTISKAKGQPREPQVYTLPPSQEEMTKN | |
| | | QVSLTCLVKGFYPSDIAVEWESNGQPENNYK | |
| | | TTPPVLDSDGSFLLYSKLTVDKSRWQEGNVF | |
| | | SCSVMHEALHNHYTQKSLSLSLGK | |
| | LC | QTVVTQEPSLTVSPGGTVTLTCRSSTGAVTT | 24 |
| | | SNYANWVQQKPGQAPRGLIGGTNKRAPGTP | |
| | | ARFSGSLLGGKAALTLSGVQPEDEAEYYCAL | |
| | | WYSNLWVFGGGTKLTVLGQPKAAPSVTLFP | |
| | | PSSEELQANKATLVCLISDFYPGAVTVAWKA | |
| | | DSSPVKAGVETTTPSKQSNNKYAASSYLSLT | |
| | | PEQWKSHRSYSCQVTHEGSTVEKTVAPTECS | |

TABLE 3
Sequences of GPRC5D binding arm of Talquetamab

| | | | SEQ |
|---|---|---|---|
| | | | ID |
| | Region | Sequence | NO: |
| GC5B596 | HCDR1 | GYTMN | 25 |
| | HCDR2 | LINPYNSDTNYAQKLQG | 26 |
| | HCDR3 | VALRVALDY | 27 |
| | LCDR1 | KASQNVATHVG | 28 |
| | LCDR2 | SASYRYS | 29 |
| | LCDR3 | QQYNRYPYT | 30 |
| | VH | QVQLVQSGAEVKKPGASVKVSCKASGYSFT | 31 |
| | | GYTMNWVRQAPGQGLEWMGLINPYNSDTNY | |
| | | AQKLQGRVTMTTDTSTSTAYMELRSLRSDD | |
| | | TAVYYCARVALRVALDYWGQGTLVTVSS | |
| | VL | DIQMTQSPSSLSASVGDRVTITCKASQNVA | 32 |
| | | THVGWYQQKPGKAPKRLIYSASYRYSGVPS | |
| | | RFSGSGSGTEFTLTISNLQPEDFATYYCQQ | |
| | | YNRYPYTFGQGTKLEIK | |
| | HC | QVQLVQSGAEVKKPGASVKVSCKASGYSFT | 33 |
| | | GYTMNWVRQAPGQGLEWMGLINPYNSDTNY | |
| | | AQKLQGRVTMTTDTSTSTAYMELRSLRSDD | |
| | | TAVYYCARVALRVALDYWGQGTLVTVSSAS | |
| | | TKGPSVFPLAPCSRSTSESTAALGCLVKDY | |
| | | FPEPVTVSWNSGALTSGVHTFPAVLQSSGL | |
| | | YSLSSVVTVPSSSLGTKTYTCNVDHKPSNT | |
| | | KVDKRVESKYGPPCPPCPAPEAAGGPSVFL | |
| | | FPPKPKDTLMISRTPEVTCVVVDVSQEDPE | |
| | | VQFNWYVDGVEVHNAKTKPREEQFNSTYRV | |
| | | VSVLTVLHQDWLNGKEYKCKVSNKGLPSSI | |
| | | EKTISKAKGQPREPQVYTLPPSQEEMTKNQ | |
| | | VSLTCLVKGFYPSDIAVEWESNGQPENNYK | |
| | | TTPPVIDSDGSIFLYSRLTVDKSRWQEGNV | |
| | | FSCSVMHEALHNHYTQKSLSLSLGK | |
| | LC | DIQMTQSPSSLSASVGDRVTITCKASQNVA | 34 |
| | | GTHVWYQQKPGKAPKRLIYSASYRYSGVPS | |
| | | RFSGSGSGTEFTLTISNLQPEDFATYYCQQ | |
| | | YNRYPYTFGQGTKLEIKKAAPSVTLFPPSS | |
| | | EELQANKATLVCLISDFYPGAVTVAWKGDS | |
| | | SPVKAGVETTTPSKQSNNKYAASSYLSLTP | |
| | | EQWKSHRSYSCQVTHEGSTVEKTVAPTECS | |

A CD3 bispecific antibody useful for the invention can be formulated as a pharmaceutical composition comprising about 1 mg/mL to about 200 mg/mL antibody, such as about 1 mg/ml, about 5 mg/ml, about 10 mg/ml, about 15 mg/ml, about 20 mg/mL, about 25 mg/mL, about 30 mg/mL, about 35 mg/mL, about 40 mg/mL, about 45 mg/mL, about 50 mg/mL, about 60 mg/mL, about 70 mg/mL, about 80 mg/mL, about 90 mg/mL, about 100 mg/mL, about 110 mg/mL, about 120 mg/mL, or any value in between, of the CD3 bispecific antibody.

The pharmaceutical compositions can further comprise one or more excipients. In some embodiments, the one or more excipients include, but are not limited to, a buffering agent, a sugar, a surfactant, a chelator, metal ion scavenger, or any combination thereof.

In some embodiments, the CD3 bispecific antibody is administered by an intravenous injection. In some embodiments, the CD3 bispecific antibody is administered by a subcutaneous injection.

The dose of the CD3 bispecific antibody given to a subject having cancer, such as multiple myeloma or plasmacytoma, is sufficient to alleviate or at least partially arrest the disease being treated (“therapeutically effective amount”) and includes from about 0.1 μg/kg to about 6000 μg/kg, e.g. about 0.3 μg/kg to about 5000 μg/kg, about 0.1 μg/kg to about 3000 μg/kg, about 0.2 μg/kg to about 3000 μg/kg, about 0.3 μg/kg to about 3000 μg/kg, about 0.6 μg/kg to about 3000 μg/kg, about 1.2 μg/kg to about 3000 μg/kg, about 19.2 μg/kg to about 3000 μg/kg, about 35 μg/kg to about 3000 μg/kg, about 80 μg/kg to about 3000 μg/kg, about 100 μg/kg to about 3000 μg/kg, about 270 μg/kg to about 3000 μg/kg, about 720 μg/kg to about 3000 μg/kg, about 0.1 μg/kg to about 1800 μg/kg, about 0.2 μg/kg to about 1800 μg/kg, about 0.3 μg/kg to about 1800 μg/kg, about 0.6 μg/kg to about 1800 μg/kg, about 1.2 μg/kg to about 1800 μg/kg, about 19.2 μg/kg to about 1800 μg/kg, about 35 μg/kg to about 1800 μg/kg, about 80 μg/kg to about 1800 μg/kg, about 100 μg/kg to about 1800 μg/kg, about 270 μg/kg to about 1800 μg/kg, about 720 μg/kg to about 1800 μg/kg, about 0.1 μg/kg to about 1500 μg/kg, about 0.2 μg/kg to about 1500 μg/kg, about 0.3 μg/kg to about 1500 μg/kg, about 0.6 μg/kg to about 1500 μg/kg, about 1.2 μg/kg to about 1500 μg/kg, about 19.2 μg/kg to about 1500 μg/kg, about 35 μg/kg to about 1500 μg/kg, about 80 μg/kg to about 1500 μg/kg, about 100 μg/kg to about 1500 μg/kg, about 270 μg/kg to about 1500 μg/kg, about 720 μg/kg to about 1500 μg/kg, about 0.1 μg/kg to about 850 μg/kg, about 0.2 μg/kg to about 850 μg/kg, about 0.3 μg/kg to about 850 μg/kg, about 0.6 μg/kg to about 850 μg/kg, about 1.2 μg/kg to about 850 μg/kg, about 19.2 μg/kg to about 850 μg/kg, about 35 μg/kg to about 850 μg/kg, about 80 μg/kg to about 850 μg/kg, about 100 μg/kg to about 850 μg/kg, about 270 μg/kg to about 850 μg/kg, about 720 μg/kg to about 850 μg/kg, about 0.1 μg/kg to about 720 μg/kg, about 0.2 μg/kg to about 720 μg/kg, about 0.3 μg/kg to about 720 μg/kg, about 0.6 μg/kg to about 720 μg/kg, about 1.2 μg/kg to about 720 μg/kg, about 19.2 μg/kg to about 720 μg/kg, about 35 μg/kg to about 720 μg/kg, about 80 μg/kg to about 720 μg/kg, about 100 μg/kg to about 720 μg/kg, about 270 μg/kg to about 720 μg/kg, about 720 μg/kg to about 720 μg/kg, about 0.1 μg/kg to about 270 μg/kg, about 0.2 μg/kg to about 270 μg/kg, about 0.3 μg/kg to about 270 μg/kg, about 0.6 μg/kg to about 270 μg/kg, about 1.2 μg/kg to about 270 μg/kg, about 19.2 μg/kg to about 270 μg/kg, about 35 μg/kg to about 270 μg/kg, about 80 μg/kg to about 270 μg/kg, about 100 μg/kg to about 270 μg/kg, about 270 μg/kg to about 270 μg/kg, about 720 μg/kg to about 270 μg/kg, about 0.1 μg/kg to about 100 μg/kg, about 0.2 μg/kg to about 100 μg/kg, about 0.3 μg/kg to about 100 μg/kg, about 0.6 μg/kg to about 100 μg/kg, about 1.2 μg/kg to about 100 μg/kg, about 19.2 μg/kg to about 100 μg/kg, about 35 μg/kg to about 100 μg/kg, about 80 μg/kg to about 100 μg/kg, about 100 μg/kg to about 100 μg/kg, about 270 μg/kg to about 100 μg/kg, about 720 μg/kg to about 100 μg/kg of the antibody. Suitable doses include, e.g., about 0.1 μg/kg, about 0.2 μg/kg, about 0.3 μg/kg, about 0.6 μg/kg, about 1.2 μg/kg, about 2.4 μg/kg, about 4.8 μg/kg, about 9.6 μg/kg, about 19.2 μg/kg, about 20 μg/kg, about 35 μg/kg, about 38.4 μg/kg, about 40 μg/kg, about 50 μg/kg, about 57.6 μg/kg, about 60 μg/kg, about 80 μg/kg, about 100 μg/kg, about 120 μg/kg, about 180 μg/kg, about 240 μg/kg, about 270 μg/kg, about 300 μg/kg, about 720 μg/kg, about 850 μg/kg, about 1000 μg/kg, about 1100 μg/kg, about 1200 μg/kg, about 1300 μg/kg, about 1400 μg/kg, about 1500 μg/kg, about 1600 μg/kg, about 1700 μg/kg, about 1800 μg/kg, about 2000 μg/kg, about 2500 μg/kg, about 3000 μg/kg, about 3500 μg/kg, about 4000 μg/kg, about 4500 μg/kg, about 5000 μg/kg, about 5500 μg/kg, about 6000 μg/kg, or any dose in between.

A fixed unit dose of the CD3 bispecific antibody can also be given, for example, 50, 100, 200, 500, or 1000 mg, or any value in between, or the dose can be based on the patient's surface area, e.g., 500, 400, 300, 250, 200, or 100 mg/m², or any value in between. Usually 1 to 8 doses, (e.g., 1, 2, 3, 4, 5, 6, 7, or 8) can be administered to treat a cancer, such as MM or plasmacytoma, but 9, 10, 11, 12, 13, 14, 15, 16, 17, 18, 19, 20, or more doses can be given.

The administration of the CD3 bispecific antibody can be repeated after one day, two days, three days, four days, five days, six days, one week, two weeks, three weeks, one month, five weeks, six weeks, seven weeks, two months, three months, four months, five months, six months, or longer. Repeated courses of treatment are also possible, as is chronic administration. The repeated administration (“cycle”) can be at the same dose or at a different dose. For example, the CD3 bispecific antibody can be administered at a first dose at weekly intervals for a certain number of weeks, followed by administration at a second dose every two weeks (i.e., bi-weekly) for an additional certain number of weeks, followed by administration at a third dose every week for an additional certain number of weeks.

The CD3 bispecific antibody can be administered by maintenance therapy, such as, e.g., once a week for a period of 6 months or more. For example, the CD3 bispecific antibody can be provided as a daily dosage in an amount of about 0.1 μg/kg to about 6000 μg/kg, e.g. about 0.2 μg/kg to about 3000 μg/kg, about 0.2 μg/kg to about 2000 μg/kg, about 0.2 μg/kg to about 1500 μg/kg, about 0.3 μg/kg to about 1500 μg/kg, about 0.6 μg/kg to about 720 μg/kg, about 1.2 μg/kg to about 270 μg/kg, about 19.2 μg/kg to about 720 μg/kg, about 35 μg/kg to about 850 μg/kg, about 270 μg/kg to about 720 μg/kg, of the antibody per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19, or 20 after initiation of treatment, or any combination thereof, using single or divided doses of every 24, 12, 8, 6, 4, or 2 hours, or any combination thereof.

In one embodiment, the CD3 bispecific antibody is administered intravenously once a week at a single dose. For example, the CD3 bispecific antibody can be administered intravenously once a week in an amount of about 0.1 μg/kg, about 0.2 μg/kg, about 0.3 μg/kg, about 0.6 μg/kg, about 1.2 μg/kg, about 2.4 μg/kg, about 4.8 μg/kg, about 9.6 μg/kg, about 19.2 μg/kg, about 20 μg/kg, about 35 μg/kg, about 38.4 μg/kg, about 40 μg/kg, about 50 μg/kg, about 57.6 μg/kg, about 60 μg/kg, about 80 μg/kg, about 100 μg/kg, about 120 μg/kg, about 180 μg/kg, about 240 μg/kg, about 270 μg/kg, about 300 μg/kg, about 720 μg/kg, about 850 μg/kg, about 1000 μg/kg, about 1100 μg/kg, about 1200 μg/kg, about 1300 μg/kg, about 1400 μg/kg, about 1500 μg/kg, about 1500 μg/kg, about 1600 μg/kg, about 1700 μg/kg, about 1800 μg/kg, or any dose in between.

In one embodiment, the CD3 bispecific antibody is administered intravenously twice a week at a single dose. For example, the CD3 bispecific antibody can be administered intravenously twice a week in an amount of about 0.1 μg/kg, about 0.2 μg/kg, about 0.3 μg/kg, about 0.6 μg/kg, about 1.2 μg/kg, about 2.4 μg/kg, about 4.8 μg/kg, about 9.6 μg/kg, about 19.2 μg/kg, about 20 μg/kg, about 35 μg/kg, about 38.4 μg/kg, about 40 μg/kg, about 50 μg/kg, about 57.6 μg/kg, about 60 μg/kg, about 80 μg/kg, about 100 μg/kg, about 120 μg/kg, about 180 μg/kg, about 240 μg/kg, about 270 μg/kg, about 300 μg/kg, about 720 μg/kg, about 850 μg/kg, about 1000 μg/kg, about 1100 μg/kg, about 1200 μg/kg, about 1300 μg/kg, about 1400 μg/kg, about 1500 μg/kg, about 1500 μg/kg, about 1600 μg/kg, about 1700 μg/kg, about 1800 μg/kg, or any dose in between.

In one embodiment, the CD3 bispecific antibody is administered intravenously at a step-up (or “priming”) dose, followed by weekly administration at a higher dose. For example, the CD3 bispecific antibody can be administered intravenously at a step-up dose of about 0.1 μg/kg, about 0.2 μg/kg, about 0.3 μg/kg, about 0.6 μg/kg, about 1.2 μg/kg, about 2.4 μg/kg, about 4.8 μg/kg, about 9.6 μg/kg, about 10 μg/kg, about 19.2 μg/kg, about 20 μg/kg, or any dose in between, followed by weekly intravenous administration at a dose of about 35 μg/kg, about 38.4 μg/kg, about 40 μg/kg, about 50 μg/kg, about 57.6 μg/kg, about 60 μg/kg, about 80 μg/kg, or any dose in between.

In one embodiment, the CD3 bispecific antibody is administered intravenously at a step-up dose, followed by administration at a higher step-up dose, followed by weekly administration at a third, higher dose. For example, the CD3 bispecific antibody can be administered intravenously at a step-up dose of about 0.1 μg/kg, about 0.2 μg/kg, about 0.3 μg/kg, about 0.6 μg/kg, about 1.2 μg/kg, about 2.4 μg/kg, about 4.8 μg/kg, about 9.6 μg/kg, about 10 μg/kg, about 19.2 μg/kg, about 20 μg/kg, or any dose in between, followed by intravenous administration at a step-up dose of about 35 μg/kg, about 38.4 μg/kg, about 40 μg/kg, about 50 μg/kg, about 57.6 μg/kg, about 60 μg/kg, about 80 μg/kg, or any dose in between, followed by weekly intravenous administration at a dose of about 80 μg/kg, about 100 μg/kg, about 120 μg/kg, about 180 μg/kg, about 240 μg/kg, about 270 μg/kg, or any dose in between.

In one embodiment, the CD3 bispecific antibody is administered intravenously at a step-up dose, followed by administration at a higher step-up dose, followed by administration at a third, higher step-up dose, followed by weekly administration at a fourth, higher dose. For example, the CD3 bispecific antibody can be administered intravenously at a step-up dose of about 0.1 μg/kg, about 0.2 μg/kg, about 0.3 μg/kg, about 0.6 μg/kg, about 1.2 μg/kg, about 2.4 μg/kg, about 4.8 μg/kg, about 9.6 μg/kg, about 10 μg/kg, about 19.2 μg/kg, about 20 μg/kg, or any dose in between, followed by intravenous administration at a step-up dose of about 35 μg/kg, about 38.4 μg/kg, about 40 μg/kg, about 50 μg/kg, about 57.6 μg/kg, about 60 μg/kg, about 80 μg/kg, or any dose in between, followed by intravenous administration at a step-up dose of about 80 μg/kg, about 100 μg/kg, about 120 μg/kg, about 180 μg/kg, about 240 μg/kg, about 270 μg/kg, or any dose in between, followed by weekly intravenous administration at a dose of about 300 μg/kg, about 720 μg/kg, about 850 μg/kg, about 1000 μg/kg, about 1100 μg/kg, about 1200 μg/kg, about 1300 μg/kg, about 1400 μg/kg, about 1500 μg/kg, about 1600 μg/kg, about 1700 μg/kg, about 1800 μg/kg, or any dose in between.

In one embodiment, the CD3 bispecific antibody is administered subcutaneously once a week at a single dose. For example, the CD3 bispecific antibody can be administered subcutaneously once a week in an amount of about 0.1 μg/kg, about 0.2 μg/kg, about 0.3 μg/kg, about 0.6 μg/kg, about 1.2 μg/kg, about 2.4 μg/kg, about 4.8 μg/kg, about 9.6 μg/kg, about 19.2 μg/kg, about 20 μg/kg, about 35 μg/kg, about 38.4 μg/kg, about 40 μg/kg, about 50 μg/kg, about 57.6 μg/kg, about 60 μg/kg, about 80 μg/kg, about 100 μg/kg, about 120 μg/kg, about 180 μg/kg, about 240 μg/kg, about 270 μg/kg, about 300 μg/kg, about 720 μg/kg, about 850 μg/kg, about 1000 μg/kg, about 1100 μg/kg, about 1200 μg/kg, about 1300 μg/kg, about 1400 μg/kg, about 1500 μg/kg, about 1500 μg/kg, about 1600 μg/kg, about 1700 μg/kg, about 1800 μg/kg, about 2000 μg/kg, about 2500 μg/kg, about 3000 μg/kg, about 3500 μg/kg, about 4000 μg/kg, about 4500 μg/kg, about 5000 μg/kg, or any dose in between.

In one embodiment, the CD3 bispecific antibody is administered subcutaneously at a step-up dose, followed by weekly administration at a higher dose. For example, the CD3 bispecific antibody can be administered subcutaneously at a step-up dose of about 10 μg/kg, about 20 μg/kg, about 35 μg/kg, about 40 μg/kg, about 50 μg/kg, about 60 μg/kg, or any dose in between, followed by weekly subcutaneously administration at a dose of about 80 μg/kg, about 100 μg/kg, about 240 μg/kg, about 300 μg/kg, or any dose in between.

In one embodiment, the CD3 bispecific antibody is administered subcutaneously at a step-up dose, followed by administration at a higher step-up dose, followed by weekly administration at a third, higher dose. For example, the CD3 bispecific antibody can be administered subcutaneously at a step-up dose of about 10 μg/kg, about 20 μg/kg, about 35 μg/kg, about 40 μg/kg, about 50 μg/kg, about 60 μg/kg, or any dose in between, followed by subcutaneously administration at a step-up dose of about 80 μg/kg, about 100 μg/kg, about 240 μg/kg, about 300 μg/kg, or any dose in between, followed by weekly subcutaneously administration at a dose of about 240 μg/kg, about 720 μg/kg, about 1100 μg/kg, about 1200 μg/kg, about 1300 μg/kg, about 1400 μg/kg, about 1500 μg/kg, about 1600 μg/kg, about 1700 μg/kg, about 1800 μg/kg, about 2000 μg/kg, about 2500 μg/kg, about 3000 μg/kg, or any dose in between.

In some embodiments, the CD3 bispecific antibody is administered for a time sufficient to achieve complete response, stringent complete response, very good partial response, partial response, minimal response or stable disease status, and can be continued until disease progression or lack of patient benefit. The disease status can be determined by any suitable method known to those skilled in the art in view of the present disclosure, including, e.g., analysis of serum and urine monoclonal protein concentrations, M-protein levels, sBCMA levels, BCMA levels, GPRC5D levels.

In some embodiments, the CD3 bispecific antibody is administered for a time sufficient to achieve complete response that is characterized by negative minimal residual disease (MRD) status. Negative MRD status can be determined by any method suitable method known to those skilled in the art in view of the present disclosure. In some embodiments, negative MRD status is determined using next generation sequencing (NGS). In some embodiments, negative MRD status is determined at 10⁻⁴ cells, 10⁻⁵ cells, or 10⁻⁶ cells.

The CD3 bispecific antibody can also be administered prophylactically in order to reduce the risk of developing cancer, such as multiple myeloma or plasmacytoma, delay the onset of the occurrence of an event in cancer progression, and/or reduce the risk of recurrence when the cancer is in remission.

In some embodiments, the therapy is a chimeric antigen receptor (CAR) or CAR-T therapy. Exemplary CARs that can be used in methods of the application are described in WO2017/025038 and WO2018/028647, the contents of which are incorporated herein by reference in their entireties.

In certain embodiments, a method of the application further comprises administering to the subject one or more other anti-cancer therapies.

The one or more other anti-cancer therapies can include, without limitation, autologous stem cell transplants (ASCT), radiation, surgery, chemotherapeutic agents, CAR-T therapies, cellular therapies, immunomodulatory agents, targeted cancer therapies, and any combination thereof.

The one or more other anti-cancer therapies can also include, without limitation, selinexor, belantamab mafodotin-blmf, isatuximab, venetoclax, lenalidomide, thalidomide, pomalidomide, bortezomib, carfilzomib, elotozumab, ixazomib, melphalan, dexamethasone, vincristine, cyclophosphamide, hydroxydaunorubicin, prednisone, rituximab, imatinib, dasatinib, nilotinib, bosutinib, ponatinib, bafetinib, saracatinib, tozasertib, danusertib, cytarabine, daunorubicin, idarubicin, mitoxantrone, hydroxyurea, decitabine, cladribine, fludarabine, topotecan, etoposide 6-thioguanine, corticosteroid, methotrexate, 6-mercaptopurine, azacitidine, arsenic trioxide and all-trans retinoic acid, and any combination thereof.

Thus, provided herein is a combination of an effective amount of a CD3 bispecific antibody, and an effective amount of other anti-cancer therapies for use in treating a hematological malignancy or a plasma cell proliferative disorder, such as MM or plasmacytoma, preferably a MM or plasmacytoma that is relapsed or refractory to a prior anti-cancer therapy.

As used herein, the terms and phrases “in combination,” “in combination with,” “co-delivery,” and “administered together with” in the context of the administration of two or more therapies or components to a subject refers to simultaneous administration, overlapping administration or subsequent administration of two or more therapies or components. “Simultaneous administration” or “simultaneously administered” refers to administration of the two or more therapies or components within the same treatment period. When two components are administered “within the same treatment period,” they can be administered in separate compositions according to their own administration schedules, as long as the periods of administration for the two components end around the same day or within a short time period, such as within 1 day, 1 week, or 1 month. “Overlapping administration” refers to administration of the two or more therapies or components not within the same overall treatment period, but with at least one overlapping treatment period. “Subsequent administration” refers to administration of the two or more therapies or components during different treatment periods, one after the other. The use of the term “in combination with” does not restrict the order in which therapies or components are administered to a subject. For example, a first therapy or component can be administered prior to, concomitantly with or simultaneously with, or subsequent to the administration of a second therapy or component.

While having described the invention in general terms, the embodiments of the invention will be further disclosed in the following examples that should not be construed as limiting the scope of the claims.

### EXAMPLES

The following examples are provided to further describe some of the embodiments disclosed herein. The examples are intended to illustrate, not to limit, the disclosed embodiments.

### Example 1

The objective of this work was to evaluate sBCMA in relapsed and/or refractory MM patients in response to treatment of teclistamab or talquetamab. Serum samples for sBCMA from relapsed and/or refractory MM patients in teclistamab and talquetamab phase 1 studies (64007957MMY1001 and 64407564MMY1001) were collected at various timepoints between baseline and cycle 4 or end of treatment, and analyzed by an electrochemiluminescence ligand binding assay. Teclistamab was administered IV once every 2 weeks (treatment doses ranging from 0.3 to 19.2 μg/kg) or weekly (treatment doses ranging from 19.2 to 720 μg/kg), or SC weekly (treatment doses ranging from 19.2 to 3000 μg/kg) for 21-day cycles. Talquetamab was administered IV once every 2 weeks (treatment doses ranging from 0.5 to 3.38 μg/kg) or weekly (treatment doses ranging from 1 to 180 μg/kg), or SC weekly (treatment doses ranging from 5 to 800 μg/kg) for 21-day cycles. 96 patients treated with teclistamab and 99 patients treated with talquetamab had evaluable data at baseline and on Cycle 3 Day 1; 147 patients in the teclistamab study and 153 patients in the talquetamab study had evaluable baseline data.

sBCMA data were quantitatively analyzed in reference to patient's response, tumor burden, and cytogenetic risk, as well as PK data. Cytogenetic risk was determined through fluorescence in situ hybridization. P values between patients with high versus standard cytogenetic risk were calculated using an unpaired 2-sample Wilcoxon test.

The criteria for response are shown in Table 4 below.

TABLE 4
Criteria for Response to MM Treatment

| Response | Response Criteria |
|---|---|
| Stringent | CR as defined below, plus |
| complete | Normal FLC ratio, and |
| Response | Absence of clonal PCs by immunohistochemistry. |
| (sCR) | immunofluorescencea or 2- to 4-color flow cytometry |
| Complete | Negative immunofixation on the serum and urine, and |
| response | Disappearance of any soft tissue plasmacytomas, and |
| (CR)* | <5% PCs in bone marrow |
| Very good | Seram and urine M-component detectable by |
| partial | immunofixation but not on electrophoresis, or |
| Response | ≥90% reduction in serum M-protein plus urine M- |
| (VGPR)* | protein <100 mg/24 hours |
| Partial | ≥50% reduction of serum M-protein and reduction in 24- |
| response | hour urinary M-protein by ≥90% or to <200 mg/24 hours |
| (PR) | If the serum and urine M-protein are not measurable, a |
| | decrease of ≥50% in the difference between involved and |
| | uninvolved FLC levels is required in place of the M-protein |
| | criteria |
| | If serum and urine M-protein are not measurable, and serum |
| | free light assay is also not measurable, ≥50% reduction in |
| | bone marrow PCs is required in place of M-protein, |
| | provided baseline bone marrow plasma cell percentage |
| | was ≥30% |
| | In addition to the above criteria, if present at baseline, |
| | a ≥50% reduction in the size of soft tissue plasmacytomas |
| | is also required. |
| Minimal | ≥25% but ≤49% reduction of serum M-protein, and |
| response | Reduction in 24-h urine M-protein by 50-89% |
| (MR) | In addition to the above criteria, if present at baseline, a |
| | 25% to 49% reduction in the size of soft tissue |
| | plasmacytomas also is required |
| Stable | Not meeting criteria for CR, VGPR, PR, MR, or PD |
| disease | |
| (SD) | |
| Progressive | Increase of 25% from lowest response value in any one of |
| disease | the following: |
| (PD)† | Serum M-component (absolute increase must be ≥0.5 g/dL) |
| | Urine M-component (absolute increase must be ≥200 mg/ |
| | 24 hours) |
| | Only in subjects without measurable serum and urine M- |
| | protein levels: the difference between involved and |
| | uninvolved FLC levels (absolute increase must be >10 |
| | mg/dL) |
| | Only in subjects without measurable serum and urine M- |
| | protein levels and without measurable disease by FLC |
| | levels, bone marrow PC percentage (absolute percentage |
| | must be ≥10%) |
| | Definite development of new bone lesions or soft tissue |
| | plasmacytomas or definite increase in the size of existing |
| | bone lesions or soft tissue plasmacytomas |
| | Development of hypercalcemia (corrected serum |
| | calcium >11.5 mg/dL) that can be attributed solely to the |
| | PC proliferative disorder |

CR = complete response;
FLC = free light chain;
IMWG = International Myeloma Working Group;
M-protein = monoclonal paraprotein;
MR = minimal response;
PC = plasma cell;
PD = progressive disease;
PR = partial response;
sCR = stringent complete response;
SD = stable disease;
VGPR = very good partial response
All response categories (CR, sCR, VGPR, PR, and PD) require 2 consecutive assessments made at any time before the institution of any new therapy; CR, sCR, VGPR, PR, and SD categories also require no known evidence of progressive or new bone lesions if radiographic studies were performed. VGPR and CR categories require serum and urine studies regardless of whether disease at baseline was measurable on serum, urine, both, or neither.
Radiographic studies are not required to satisfy these response requirements. Bone marrow assessments need not be confirmed. For PD, serum M-component increases of more than or equal to 1 g/dL are sufficient to define relapse if starting M-component is ≥5 g/dL.
*Clarifications to IMWG criteria for coding CR and VGPR in subjects in whom the only measurable disease is by serum FLC levels: CR in such subjects indicates a normal FLC ratio of 0.26 to 1.65 in addition to CR criteria listed above. VGPR in such subjects requires a >90% decrease in the difference between involved and uninvolved FLC levels.
†Clarifications to IMWG criteria for coding PD: Bone marrow criteria for PD are to be used only in subjects without measurable disease by M-protein and by FLC levels; “25% increase” refers to M-protein, FLC, and bone marrow results, and does not refer to bone lesions, soft tissue plasmacytomas, or hypercalcemia and the “lowest response value” does not need to be a confirmed value.
^{a}Presence/absence of clonal cells is based upon the kappa/lambda ratio. An abnormal kappa/lambda ratio by immunohistochemistry or immunofluorescence requires a minimum of 100 plasma cells for analysis. An abnormal ratio reflecting presence of an abnormal clone is kappa/lambda of >4:1 or <1:2.
Clinical Relapse
Clinical relapse is defined using the definition of clinical relapse in IMWG criteria (Durie 2006; Kumar 2016, Rajkumar 2011). In IMWG criteria, clinical relapse is defined as requiring one or more of the following direct indicators of increasing disease or end-organ dysfunction that are considered related to the underlying plasma cell proliferative disorder:
1. Development of new soft tissue plasmacytomas or bone lesions on skeletal survey, MRI, or other imaging
2. Definite increase in the size of existing plasmacytomas or bone lesions. A definite increase is defined as a 50% (and at least 1 cm) increase as measured serially by the sum of the products of the cross-diameters of the measurable lesion
3. Hypercalcemia (>11.5 mg/dL; >2.875 mM/L)
4. Decrease in hemoglobin of more than 2 g/dL (1.25 mM) or to less than 10 g/dL
5. Rise in serum creatinine by more than or equal to 2 mg/dL (≥177 mM/L)
6. Hyperviscosity
In some subjects, bone pain may be the initial symptom of relapse in the absence of any of the above features. However, bone pain without imaging confirmation is not adequate to meet these criteria in studies.

Patients with sCR, CR, VGPR and PR were categorized as responders, and patients with MR, SD and PD were considered non-responders.

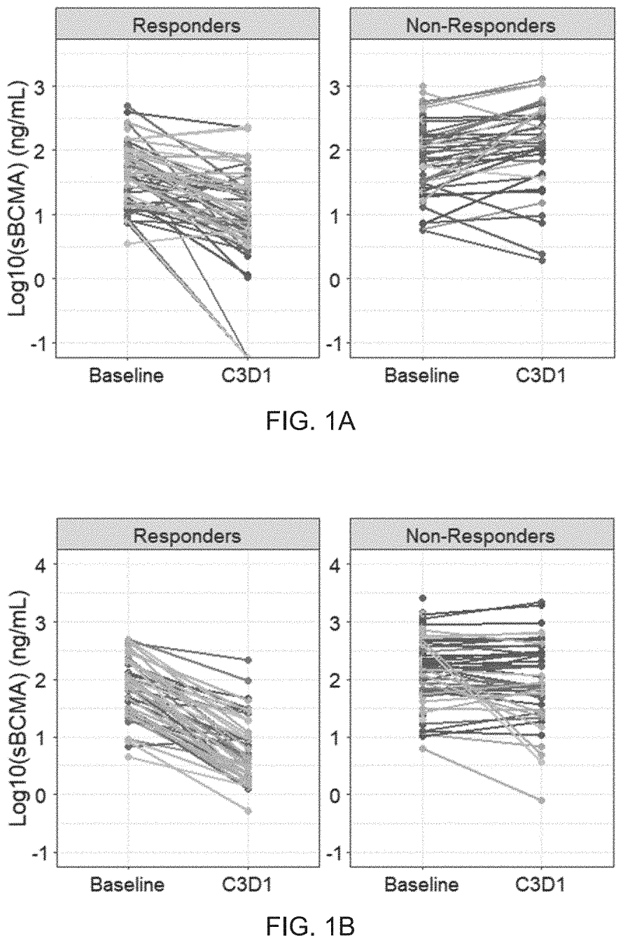

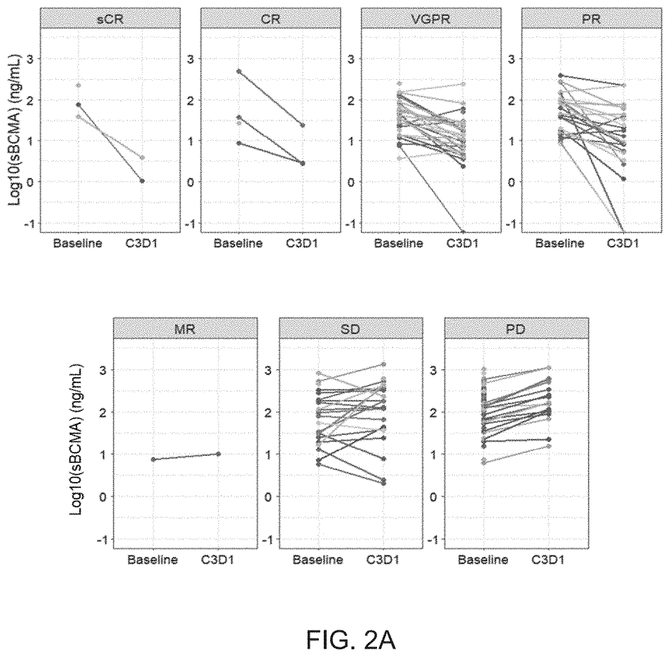

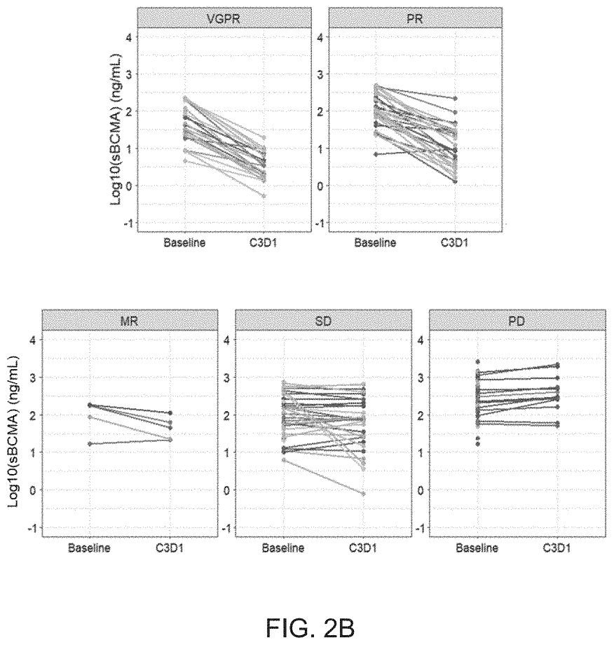

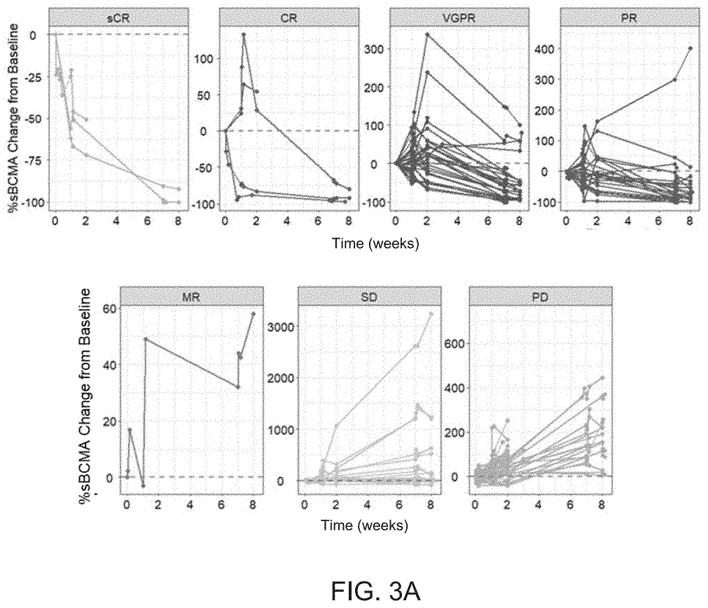

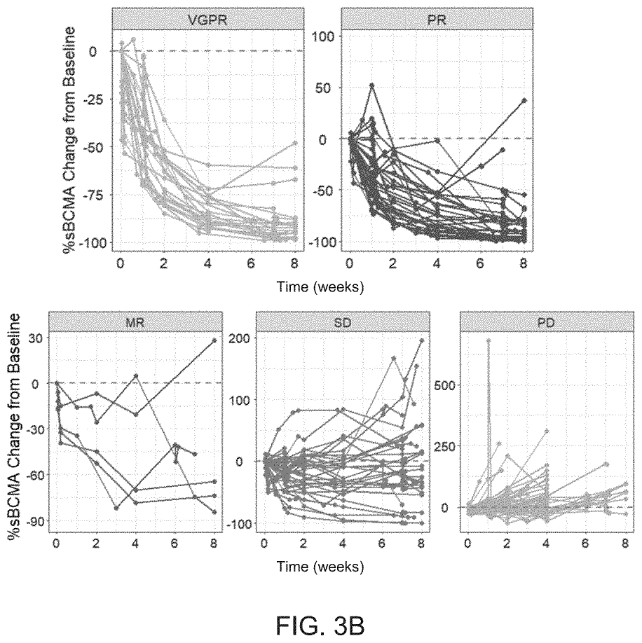

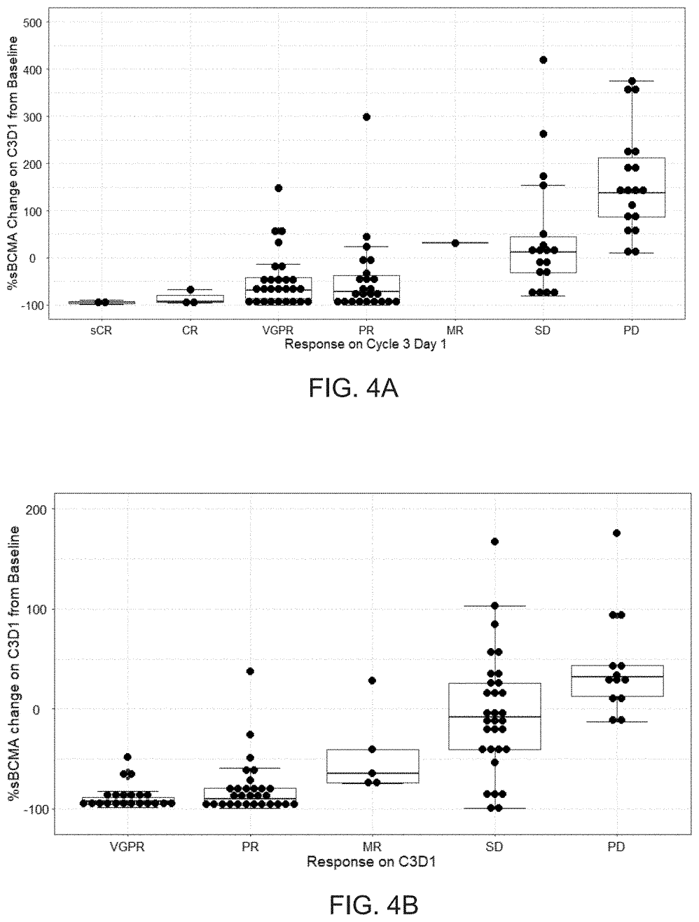

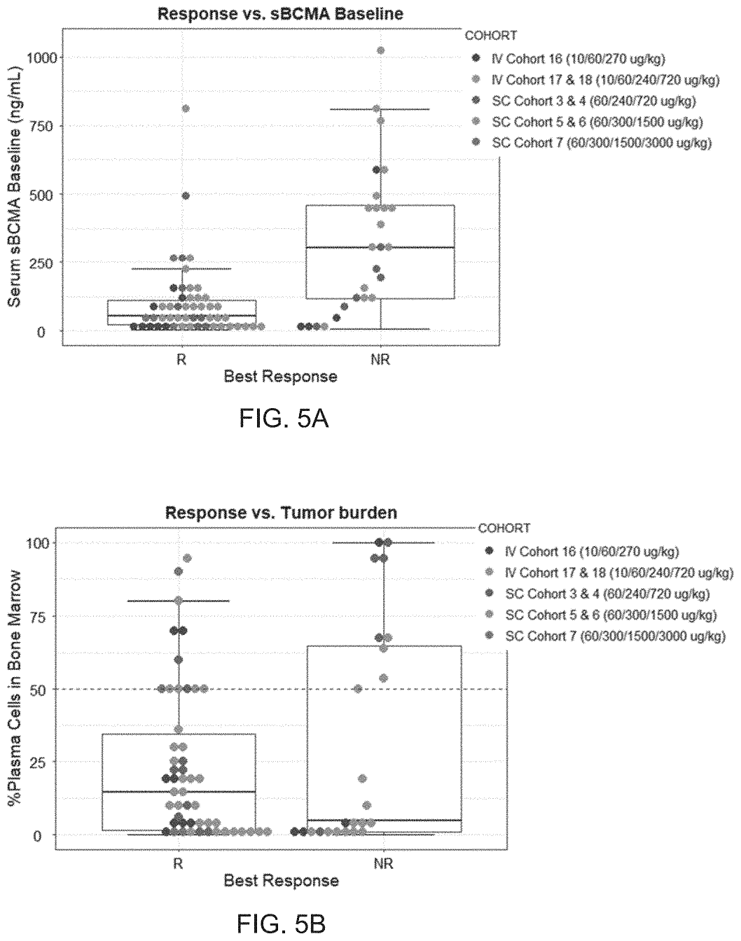

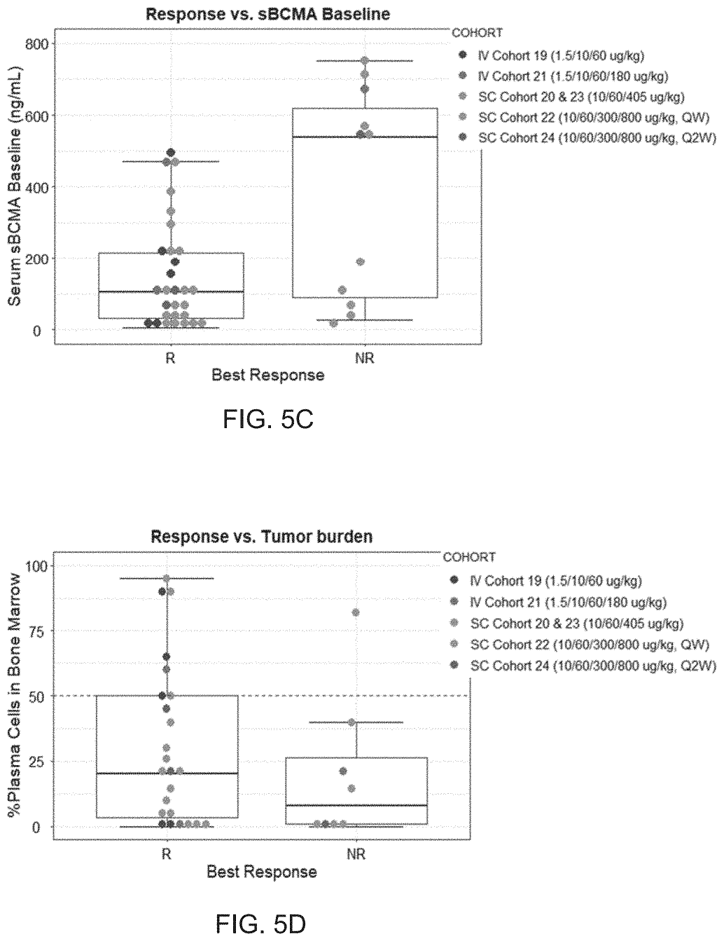

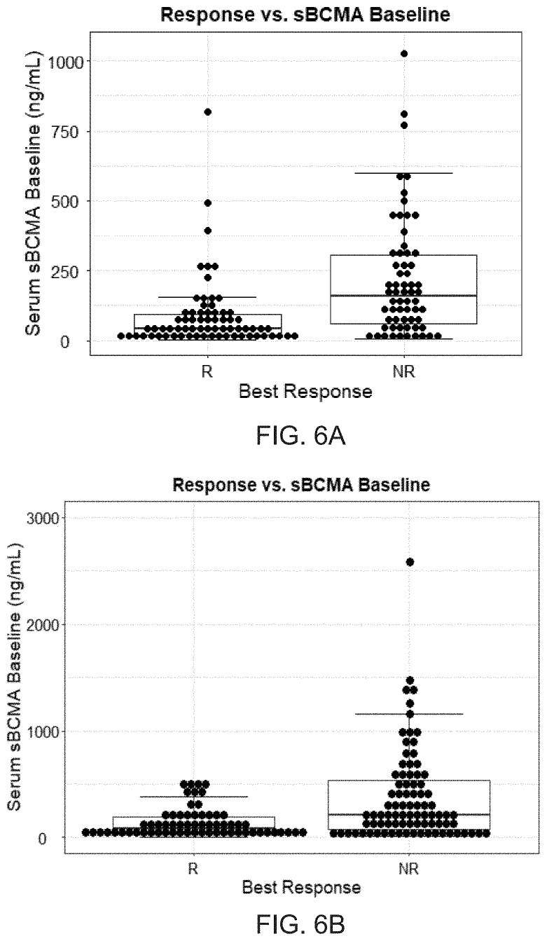

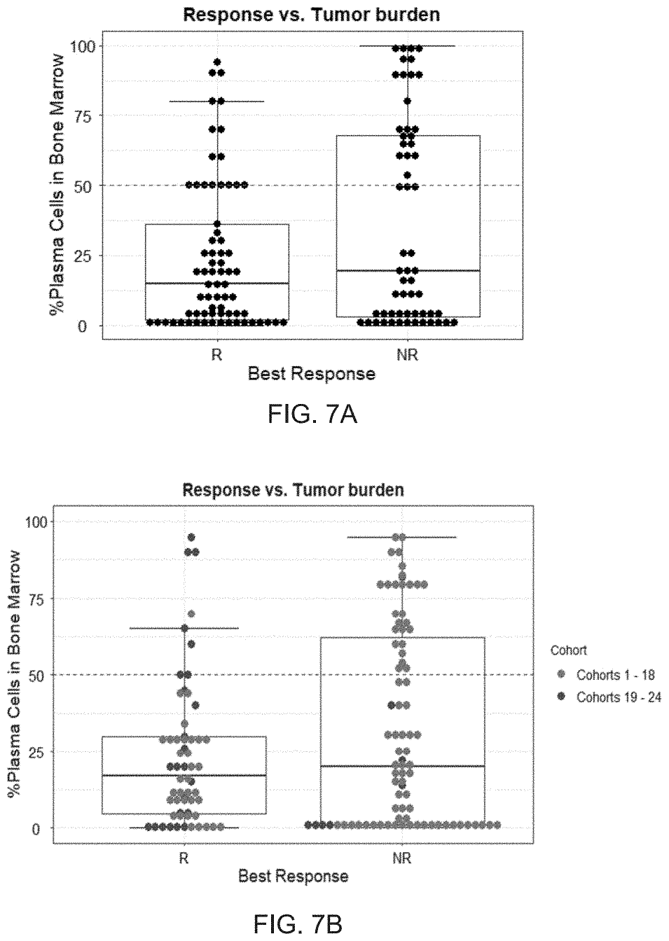

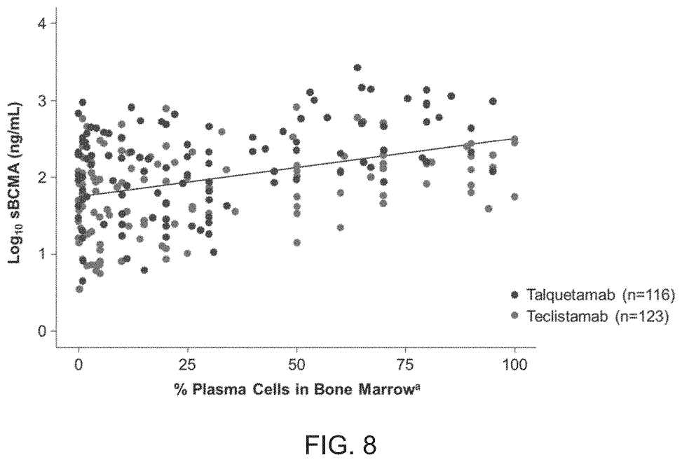

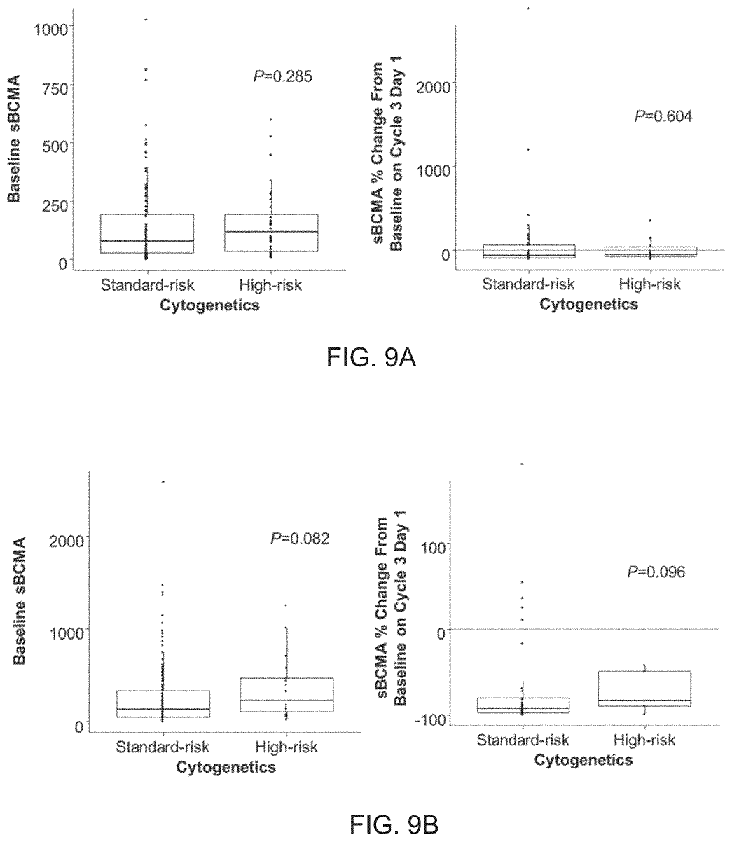

The results showed that teclistamab and talquetamab modulated levels of sBCMA in patients with high and low frequency of tumor plasma cells (TPCs), as well as in high and low risk cytogenetic groups (FIG. 9). In cycle 3, a majority of the responders had reduction in sBCMA, 88% (50 out of 57) for teclistamab and 98% (49 out of 50) for talquetamab, compared to baseline. On the contrary, non-responders (progressive disease, stable disease or minimal response) showed an increase in sBCMA, 80% (33 out of 41) for teclistamab and 49% (24 out of 49) for talquetamab, from baseline (FIGS. 1-3). Patients with deep responses tended to have higher magnitude of sBCMA reduction compared to others (FIG. 4). Soluble BCMA at baseline correlated with % bone marrow TPCs (FIG. 8). The majority of patients with plasmacytoma (limited data) seemed to have high sBCMA, suggesting sBCMA could be a comprehensive marker for tumor burden (FIGS. 5-7). Teclistamab preliminary population pharmacokinetic analysis showed that sBCMA did not appear to impact teclistamab exposure, suggesting that sBCMA was not acting as a sink for teclistamab. In conclusion, Teclistamab and Talquetamab induced changes in levels of sBCMA that correlated with clinical activity, further supporting that sBCMA is a surrogate marker of myeloma tumor burden, and a valuable marker for response in MM patients.

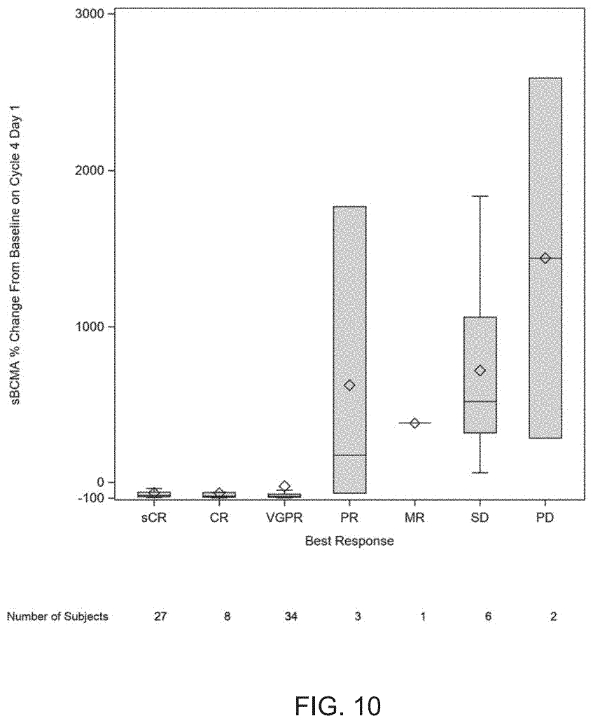

Following teclistamab at RP2D, a rapid decrease in sBCMA was observed in a majority of the responders (PR or better) within the first month of treatment. A majority of responders had a decrease in sBCMA on Cycle 2 Day 1 (40 of 59 subjects [67.8%]), and a majority of non-responders had an increase in sBCMA on Cycle 2 Day 1 (27 of 28 subjects [96.4%]) compared with baseline values. Responders to teclistamab also showed a trend of sBCMA reduction over time. On Cycle 4 Day 1, a majority of responders had a decrease in sBCMA (63 of 72 subjects [87.5%]), and all non-responders had an increase in sBCMA (9 of 9 subjects [100%]; fewer non-responders provided data on Cycle 4 Day 1 due to early treatment discontinuation. In addition, a greater reduction in sBCMA was observed in subjects with deeper responses to teclistamab (FIG. 10).

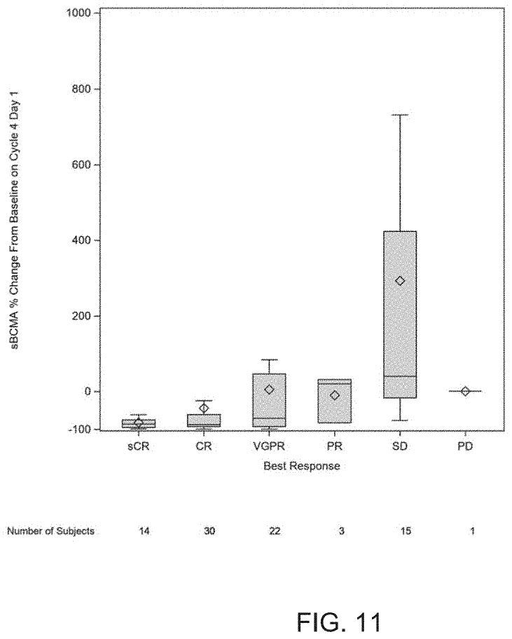

Following teclistamab IV or SC administration in Phase 1, a majority of responders had a decrease in sBCMA on Cycle 4 Day 1 (54 of 69 subjects [78.3%]), and a majority of non-responders had an increase in sBCMA on Cycle 4 Day 1 (10 of 16 subjects [62.5%]) compared with baseline values. Additionally, a greater reduction in sBCMA was observed in subjects with deeper responses to teclistamab (FIG. 11).

The possible effect of baseline sBCMA on teclistamab PK was investigated in the population PK analysis. The results suggested that baseline sBCMA did not affect teclistamab serum concentrations and was not a significant covariate of teclistamab PK.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

We claim:

1. A method of monitoring a progression of multiple myeloma in a subject, comprising:
(a) measuring a level of sBCMA in a blood sample obtained from the subject; and
(b) comparing the level of sBCMA to a reference sBCMA level, wherein the reference sBCMA level is measured from a control blood sample obtained from the subject before the blood sample of (a) is obtained from the subject;
wherein an increase in the level of sBCMA compared to the reference sBCMA level indicates one or more of an increased tumor burden or a disease progression, and a decrease in the level of sBCMA compared to the reference sBCMA level indicates one or more of a decreased tumor burden or lack of disease progression.

2. A method of determining a response to a therapy against multiple myeloma in a subject, comprising:
(a) treating the subject with the therapy;
(b) measuring a level of sBCMA in a blood sample obtained from the subject after the treating of (a); and
(c) comparing the level of sBCMA to a reference sBCMA level, wherein the reference sBCMA level is measured from a control blood sample obtained from the subject before the treating of (a);
wherein a decrease in the level of sBCMA compared to the reference sBCMA level indicates the subject is responsive to the therapy, and an increase or no change in the level of sBCMA compared to the reference sBCMA level indicates the subject is not responsive to the therapy.

3. The method of claim 2, further comprising treating the subject with a second therapy against multiple myeloma if the level of sBCMA indicates the subject is not responsive to the therapy.

4. A method of treating multiple myeloma or plasmacytoma in a subject in need thereof, comprising:
(a) measuring a level of sBCMA in a blood sample obtained from the subject;
(b) comparing the level of sBCMA to a reference sBCMA level to measure a tumor burden of the subject; and
(c) administering a therapy to the subject based on the tumor burden measured in (b).

5. The method of claim 4, further comprising treating the subject with a therapy against multiple myeloma or plasmacytoma before the blood sample is obtained from the subject, wherein the reference sBCMA level is measured from a control blood sample obtained from the subject before the subject is treated with the therapy, and the treatment comprises:
(a) continuing treating the subject with the therapy if the level of sBCMA measured in claim 4 (a) is lower than the reference sBCMA level, or
(b) treating the subject with a second therapy against multiple myeloma or plasmacytoma if the level of sBCMA is the same or higher than the reference sBCMA level.

6. A method of assessing response to teclistamab or talquetamab in a subject with multiple myeloma or plasmacytoma, comprising:
(a) treating the subject with teclistamab or talquetamab;
(b) measuring a level of sBCMA in a blood sample obtained from the subject after the treating of (a); and
(c) comparing the level of sBCMA to a reference sBCMA level, wherein the reference sBCMA level is measured from a control blood sample obtained from the subject before the treating of (a);
wherein a decrease in the level of sBCMA compared to the reference sBCMA level indicates the subject is responsive to teclistamab or talquetamab, and an increase or no change in the level of sBCMA compared to the reference sBCMA level indicates the subject is not responsive to teclistamab or talquetamab.

7. The method of claim 6, further comprising treating the subject with a second therapy against multiple myeloma or plasmacytoma if the level of sBCMA indicates the subject is not responsive to teclistamab or talquetamab.

8. The method of claim 6 wherein the blood sample is obtained from the subject from about 4 to about 16 weeks after the subject is treated with the therapy.

9. The method of claim 2 wherein the therapy comprises a CD3 bispecific antibody.

10. The method of claim 6 wherein the therapy comprises intravenously administering to the subject about from about 38 μg/kg to about 720 μg/kg per dose of teclistamab.

11. The method of claim 6 wherein the therapy comprises subcutaneously administering to the subject from about 80 μg/kg to about 3000 μg/kg per dose of teclistamab.

12. The method of claim 6 wherein the therapy comprises intravenously administering to the subject from about 0.5 μg/kg to about 180 μg/kg per dose of talquetamab.

13. The method of claim 6 wherein the therapy comprises subcutaneously administering to the subject from about 5 μg/kg to about 800 μg/kg per dose of talquetamab.

14. The method of claim 6 wherein the therapy is administered bi-weekly or weekly.

15. The method of claim 3 wherein the second therapy comprises one or more of autologous stem cell transplants (ASCT), radiation, surgery, chemotherapeutic agents, CAR-T therapies, cellular therapies, immunomodulatory agents, targeted cancer therapies, or combinations thereof.

16. The method of claim 11 wherein the subject has relapsed and/or refractory multiple myeloma.

17. The method of claim 13 wherein the subject has relapsed and/or refractory multiple myeloma.

18. The method of claim 6 wherein the blood sample is serum, whole blood, or plasma, preferably serum.

19. The method of claim 6 wherein the level of sBCMA in the blood sample is measured using an electrochemiluminescence ligand binding assay, an enzyme-linked immunosorbent assay (ELISA), or mass spectrometry.
